(19) United States
(12) Patent Application Publication
(10) Pub. No.: US 2005/0226838 A1
(43) Pub. Date: Oct. 13, 2005

(54) Hair treatment compositions containing N-hydroxy-alkyl-O-benzyl chitosans and methods of using same

(76) Inventors: Thomas Krause, Damstadt (DE); Jan Baumeister, Farvagny-le-Grand (CH); Dirk Weber, Marly (CH); Guenther Lang, Reinheim (DE); Angelika Beyer, Waldaschaff (DE); Ellen Florig, Grasellenbach (DE); Klaus Gaenger, Pfungstadt (DE); Hartmut Schiemann, Huenfeld (DE)

Correspondence Address: STRIKER, STRIKER & STENBY, 103 EAST NECK ROAD, HUNTINGTON, NY, 11743, US

(21) Appl. No.: 11/100,225

(22) Filed: Apr. 6, 2005

(30) Foreign Application Priority Data
Apr. 8, 2004 (DE) 10 2004 017431.8

(51) Int. Cl. A61K 7/06; A61K 7/11

(52) U.S. Cl. 424/70.13

(57) ABSTRACT

The hair treatment composition contains preferably from 0.01 to 20 percent by weight of at least one N-hydroxyalkyl-O-benzyl chitosan and from 0.01 to 20 percent by weight of at least one other hair treatment effective ingredient. The at least one N-hydroxyalkyl-O-benzyl chitosan has at least one hydroxylalkyl group, preferably a hydroxyethyl, hydroxypropyl or hydroxybutyl group, and has from 2 to 20 carbon atoms. Various methods of treating hair with hair treatment compositions containing one or more of the N-hydroxyalkyl-O-benzyl chitosans are described.

### BACKGROUND OF THE INVENTION

The subject matter of the present invention concerns hair treatment compositions containing N-hydroxyalkyl-O-benzyl chitosans and methods of treating the hair with these compositions.

A pleasant outer appearance is always considered very important. The hair and the hairstyle play an important role. Hair treatment products are used to improve personal appearance or image. These products generally contain a number of individual substances or ingredients, which fulfill different purposes within the formulation. Polymers with the most different activity and function are among the many ingredients of cosmetic products. For example, polymers can have thickening action, preservative action, film-forming activity, hair-fixing action, hair care or conditioning action on the hair. They can improve the product consistency, and the deposit and adherence of effective ingredients on the hair or advantageously modify the properties of the remaining ingredients. There is a special need for effective ingredients, which are useable because of improved properties and which have an especially good compatibility with as many other cosmetic effective and additive ingredients as possible and in as many different product lines as possible. Polymers, which are based on naturally grown raw materials and which have satisfactory biodegradability, are especially preferred.

Derivatives of chitin and chitosans are already known as well as their use in hair treatment compositions. These derivatives can include, for example, hydroxypropyl chitosan (EP 0 192 925, EP 0 224 045), hydroxybutyl chitosan (WO 87/06461), alkylhydroxypropyl chitosan (WO 86/4590), N-hydroxypropyl-isopropylether chitosan (WO 88/05790), N-hydroxybutyl chitosan (WO 87/6461), N-hydroxyethyl chitosan (DE 36 02 402) and hydroxyalkyl chitin (EP 0 806 435). Also O-benzyl-N-hydroxyalkyl chitosans are disclosed in EP 0 300 234 for use in nail polish. N,O-substituted Chitin and Chitosan derivatives are known for use, among others, in cosmetic compositions. O-benzyl-N-hydroxyalkyl chitosan is explicitly mentioned in this reference for nail polish and is described as disadvantageous for other cosmetic preparations. Chitosan and Chitosan derivatives are usually dissolved by protonation or salt formation. This has the disadvantage that compatibility with anionic ingredients, such as anionic polymers or anionic surfactants, is impaired and hair care and hair-fixing action is reduced. The currently known chitosan derivatives have only limited application possibilities because of their solubility properties or on account of incompatibilities. They are still not entirely satisfactory in their hair care properties.

### SUMMARY OF THE INVENTION

It is an object of the present invention to provide effective ingredients for hair treatment with good hair cosmetic properties and as wide as possible an application spectrum based on good solubility and compatibility properties.

One such class of effective ingredients was found to be the N-hydroxyalkyl-O-benzyl chitosans defined hereinbelow. These compositions are advantageously useable in many different hair cosmetic agents and combined advantageously with a number of other hair cosmetic effective and additive ingredients.

Thus the subject matter of the present invention is a method of treating hair using N-hydroxyalkyl-O-benzyl chitosans. N-hydroxyalkyl-O-benzyl chitosans are polymers, which are derived from chitosan which has at least one amino group, which is substituted with at least one hydroxyalkyl or poly-(hydroxyalkyl) group, which has at least one benzyl ether group. The hydroxyalkyl unit has one or more hydroxy groups and preferably 2 to 20 carbon atoms. C₂- to C₄-alkyl groups with one hydroxy group, for example hydroxyethyl, hydroxypropyl and hydroxybutyl, especially hydroxypropyl, are especially preferred. Mixtures of N-hydroxyalkyl-O-benzyl chitosans with hydroxyalkyl groups of different chain length are used. Also one and the same N-hydroxyalkyl-O-benzyl chitosan can be substituted with hydroxyalkyl groups of different chain length.

Suitable N-hydroxyalkyl-O-benzyl chitosans are obtained by N-hydroxyalkylation with an alkylene oxide and at the same time or subsequently O-benzylizing with a reactive benzyl compound. A suitable synthesis method is described in EP 0 300 234. N-hydroxyalkyl chitosans are obtained by reaction of a chitosan with at least one alkylene oxide, e.g. ethylene oxide, propylene and/or butylenes oxide, preferably propylene oxide, in the present of a basic catalyst (N-alkoxylation). N-hydroxyalkyl-O-benzyl chitosans themselves are formed by reacting N-hydroxyalkyl chitosans with at least one reactive benzyl compound, e.g. benzyl halides, preferably benzyl chloride (O-benzylation). The making of this compound, as described in EP 0 300 234, is also performed as a one-step process. The basic chitosan can be a completely or partially deacetylated chitin. Chitosan is made from chitin obtained from the shell residues of crustaceans, which is an economical and natural raw material, which is available in large quantities. Suitable chitosans are commercially available. The molecular weights of the chitosans can be distributed over a wide spectrum, e.g. from 20,000 to about 5 million, from 30,000 to 1,000,000, preferably from 100,000 to 800,000, g/mol. The deacetylation degree amounts to e.g. 10 to 99%, 60 to 95%, preferably from 70 to 90%.

For example, N-hydroxyalkyl-O-benzyl chitosans are suitable, which have at least one unit of the general formula (I): 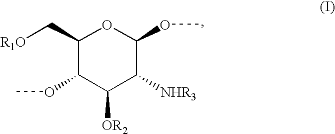
wherein R¹ and R², independently of each other, denote a -(AO)ₙ—H group, A denotes a C₂— to C₂₀—, preferably a C₂— to C₄—, alkylene group; n is a number greater than or equal to zero and R³ denotes H, —CH₂Ph or —CO—CH₃, with the proviso that n is greater than 0 and R³ equals —CH₂Ph for at least one unit and/or that n is greater than zero and R³ equals H or —CO—CH₃ for at least one first unit and n equals zero and R³ equals —CH₂Ph for at least one second unit.

The hydroxyalkylation degree is e.g. in a range of from 0.1 to 10, preferably greater than 1 to 8, especially from 1.5 to 6. The acetylation degree is preferably in a range from 0 to 0.6. The benzylation degree is preferably in a range from 0.1 to 4, preferably from 0.2 to 2. The polymerization degree is preferably in a range from 50 to 5,000. The molecular weight is preferably in a range from 5,000 to 2,000,000, especially in a range from 20,000 to 1,000,000 or 300,000 to 700,000, g/mol. Suitable N-hydroxyalkyl-O-benzylchitosans are especially those, which are made from chitosan by reaction with 2 to 7 equivalents of alkylene oxide (preferably propylene oxide) and 0.25 to 1.5 equivalents of benzyl chloride per glucosamine unit.

The subject matter of the invention includes hair treatment composition containing
(A) at least one of the above-described N-hydroxyalkyl-O-benzylchitosans, and
(B) at least one other hair treatment active or effective ingredient.

Hair treatment effective ingredients include e.g. hair care, hair protective, hair repairing, hair cleansing, hair dyeing, hair fixing or permanent shaping or hair smoothing effective ingredients, which increase hair luster or change the hair shape. The effective ingredients are preferably hair care surfactants, especially cationic or cation-active surfactants, hair-cleaning surfactants, hair care oils and waxes, hair care polymers, light protective agents, oxidation hair dye precursors, direct-dyeing dye compounds, hair dye pigments, oxidizing agents and keratin-reducing substances.

The N-hydroxyalkyl-O-benzylchitosan (A) is contained in the composition according to the invention preferably in an amount of 0.01 to 20, especially preferably from 0.1 to 10, and most especially preferably in an amount of 0.2 to 5 percent by weight. The effective ingredient (B) is contained in the composition according to the invention in an amount of 0.01 to 20, especially preferably of from 0.05 to 10, and most especially preferably from 0.1 to 5 percent by weight.

The hair treatment compositions according to the invention can be compositions for cleaning and caring for hair, such as shampoos, hair care compositions, hair rinses, tip liquids, hair oils, brillantines, which can be applied in different application forms, e.g. as leave on or as rinse-off products. They can be permanent, semi-permanent or temporary hair dye composition, e.g. oxidative hair dye compositions or non-oxidative hair coloring compositions or hair bleaching compositions. They can be permanent shaping compositions, e.g. in the form of mild alkaline or acidic permanent wave compositions, hair curling compositions or permanent wave fixing compositions as well as compositions for temporarily shaping and/or stabilizing a hairdo (styling composition), e.g. hair sprays, hair lacquers, fixing lotions, fixing foams, hair gels, hair waxes, styling creams, etc.

The chitosan derivatives used up to now for hair treatment were only poorly worked into hair care preparations. In contrast if one includes the N-hydroxyalkyl-O-benzylchitosans according to the invention in a typical hair care composition, e.g. care foam and shampoo, one obtains a stable formulation. The N-hydroxyalkyl-O-benzyl chitosans are characterized by good spreadability on the hair. The hair treated according to the invention has an improved volume effect and good feel and combability in the wet and dry state. Also stable formulations are obtained for use in typical hair styling compositions, such as hair sprays. Especially a combination of N-hydroxyalkyl-O-benzylchitosans with hair-fixing polymers leads to improved luster and feel of the treated hair besides hair-fixing.

In one embodiment the composition according to the invention further comprises a polymer with anionic or anionizable groups in an amount of preferably from 0.01 to 20 percent by weight or from 0.05 to 10 percent by weight, especially preferably from 0.1 to 5 percent by weight, as a hair-fixing or hair-care effective ingredient. Anionizable groups are understood to be acid groups, such as carboxylic acid, sulfuric acid or phosphoric acid groups, which can be deprotonated by means of a conventional base, such as an organic amine or alkali metal or alkaline earth metal hydroxide.

The anionic polymers can be partially or completely neutralized with a basic neutralization agent. Preferably they are present in 50 to 100% neutralized form, especially preferably 70 to 100%. Organic or inorganic bases suitable for cosmetic purposes can be used as neutralization agents. For example, suitable bases include amino alcohols, such as aminomethylpropanol (AMP), triethanolamine or monoethanolamine. However ammonia, NaOH and other bases are also suitable.

The anionic polymers can be homopolymers or copolymers with monomer units containing acid groups on a synthetic or natural basis, which are copolymerized as needed with comonomers, which contain no acid groups. A sulfuric acid group, a phosphoric acid group and a carboxylic acid group can be used as the acid group. The carboxylic acid group is particularly preferred. Suitable monomers containing acid groups include e.g. acrylic acid, methacrylic acid, crotonic acid, maleic acid, maleic acid anhydride, maleic acid monoesters, especially the mono-C₁ to C₇-alkyl esters of maleic acid, aldehydocarboxylic acids or ketocarboxylic acids. The comonomers not substituted with acid groups include, e.g., acryl amide, methacrylamide, alkyl- and dialkylacrylamides, alkyl- and dialkylmethacrylamides, alkylacrylates, alkylmethacrylates, vinyl caprolactone, vinyl pyrrolidone, vinyl ester, vinyl alcohol, propylene glycol or ethylene glycol, amine-substituted vinyl monomers, such as dialkylamino-alkylacrylates, dialkylaminoalkyl-methacrylates, monoalkylaminoalkylacrylates and monoalkylaminoalkylmethacrylates, in which the alkyl groups of these monomers are preferably C₁- to C₇-alkyl groups, especially preferably C₁- to C₃-alkyl groups.

Suitable polymers with acid groups are especially homopolymers of acrylic acid or methacrylic acid, which are not cross-linked or cross-linked with polyfunctional agents, copolymers of acrylic acid or methacrylic acid with monomers selected from the group consisting of acrylic acid or methacrylic acid esters, acrylamide, methacrylamide and vinyl pyrrolidones, homopolymers of crotonic acid and copolymers of crotonic acid with monomers selected from the group consisting of vinyl esters, acrylic acid esters, methacrylic acid esters, acrylamides and methacrylamides. A suitable natural polymer is, for example, shellac.

Preferred polymers with acid groups are: terpolymers of acrylic acid, alkyl acrylate and N-alkylacrylamide (INCI-name: acrylates/acrylamide copolymer), especially terpolymers of acrylic acid, ethyl acrylate and N-t-butyl-acrylamide; cross-linked or uncrosslinked vinyl acetate/crotonic acid copolymer (INCI name: VA/crotonates copolymer); copolymers of one or more C₁- to C₅-alkylacrylates, especially C₂- to C₄-alkylacrylates and at least one monomer selected from acrylic acid or methacrylic acid (INCI name:acrylates copolymer), e.g. terpolymers from tert.-butylacrylate, ethyl acrylate and methacrylic acid; sodium polystyrene sulfonate; vinyl acetate/crotonic acid/vinyl alkanoate copolymers, e.g. copolymers of vinyl acetate, crotonic acid and vinyl propionate; copolymers of vinyl acetate, crotonic acid and vinyl neodecanoate (INCI names: VA/crotonates/vinyl propionate copolymer, VA/crotonates/vinyl neodecanoate copolymer); aminomethyl propanol-acrylate copolymers; copolymers of vinyl pyrrolidone and at least one other monomer selected from the group consisting of acrylic acid, methacrylic acid, and, as needed, acrylic acid esters and methacrylic acid esters; copolymers of methyl vinyl ether and maleic acid monoalkyl esters (INCI name: ethyl ester of PVM/MA copolymer, butyl ester of PVM/MA copolymer); aminomethylpropanol salts of copolymers of allyl methacrylate and at least one other monomer selected from the group consisting of acrylic acid, methacrylic acid, and, as needed, acrylic acid esters and methacrylic acid esters; cross-linked copolymers of ethyl acrylate and methacrylic acid; copolymers of vinyl acetate, mono-n-butylmaleate and isobornyl acrylate; copolymers of two or more monomers selected from the group consisting of acrylic acid, methacrylic acid, and, as needed, acrylic acid esters and methacrylic acid esters; copolymers of octyl acrylamide and at least one monomer selected from the group consisting of acrylic acid, methacrylic acid, and, as needed, acrylic acid esters and methacrylic acid esters; polyesters of diglycol, cyclohexanedimethanol, isophthalic acid and sulfoisophthalic acid, in which the alkyl groups of the above-named polymers preferably contain 1, 2, 3 or 4 carbon atoms.

In other embodiments of the invention the composition can further comprise a zwitterionic and/or amphoteric polymer in an amount of preferably from 0.01 to 20 percent by weight or from 0.05 to 10 percent by weight, especially preferably from 0.1 to 5 percent by weight, as a hair-fixing or hair-care effective ingredient. Zwitterionic polymers have at least one anionic charge and at least one cationic charge at the same time. Amphoteric polymers have at least one acid group (e.g. carboxylic acid or sulfuric acid group) and at least one base group (e.g. amine group). The sulfuric acid groups can be deprotonated by a common base, e.g. an organic amine or an alkali metal or alkaline earth metal hydroxide.

Preferred zwitterionic or amphoteric polymers are: Copolymers formed from alkylacrylamides, alkylaminoalkylmethacrylate and two or more monomers selected from acrylic acid, methacrylic acid, and, as needed, acrylic acid esters and methacrylic acid esters, especially copolymers of octylacryl amide, acrylic acid, butylaminoethylmethacrylate, methylmethacrylate and hydroxypropylmethacrylate (INCI name: octylacrylamide/acrylates/butylaminoethyl methacrylate copolymer); copolymers, which are formed from at least one first type of monomer, which has quaternary amino groups and at least one second type of monomer, which has acid groups; copolymers of fatty alcohol acrylates, alkylaminoxide methacrylates and at least one monomer selected from acrylic acid, methacrylic acid, and, as needed, acrylic acid esters and methacrylic acid esters, especially copolymers of lauryl acrylate, stearyl acrylate, ethyl-amine oxide methacrylate and at least one monomer selected from acrylic acid, methacrylic acid and esters thereof as needed; copolymers of methacryloyl-ethylbetaine and at least one monomers selected from methacrylic acid and methacrylic acid esters; copolymers of acrylic acid, methacrylate and methacrylamido-propyltrimethylammonium chloride (INCI: polyquaternium-47); copolymers made from acrylamidopropyltrimethylammonium chloride and acrylates or copolymers made from acrylamide, acrylamido-propyltrimethylammonium chloride, 2-amidopropyl-acrylamide sulfonate and dimethylaminopropylamine (INCI: Polyquaternium-43); and oligomers or polymers made from quaternary crotonic betaines or their esters.

In other embodiments of the invention the composition can further comprise a cationic polymer, i.e. a polymer with cationic or cationizable groups, especially primary, secondary or tertiary or quaternary amine groups, in an amount of preferably from 0.01 to 20 percent by weight or from 0.05 to 10 percent by weight, especially preferably from 0.1 to 5 percent by weight, as hair care or hair-fixing effective ingredients. The cationic charge density amounts to preferably 1 to 7 meq/g.

Suitable cationic polymers preferably contain quaternary amine groups. The cationic polymers can be homopolymers or copolymers, which contain quaternary nitrogen groups in the polymer chain or preferably as substituents on one or more monomers. The monomers containing ammonium groups can be copolymerized with non-cationic monomers. Suitable cationic monomers are unsaturated, radical polymerizable compounds, which contain at least one cationic group, especially ammonium-substituted vinyl monomers include, e.g., trialkylmethacryloxyalkyl ammonium, trialkylacryloxyalkyl ammonium, dialkyldiallyl ammonium and quaternary vinyl ammonium monomers with cyclic, cationic nitrogen containing groups, such as pyridinium, imidazolium or quaternary pyrrolidone, e.g. alkylvinylimidazolium, alkylvinylpyridinium, or alkylvinylpyrrolidone salts. The alkyl groups are preferably lower alkyl groups, especially C₁- to C₇-alkyl groups, preferably C₁- to C₃-alkyl groups.

The monomers containing ammonium groups can be copolymerized with non-cationic monomers. Suitable comonomers are for example acryl amide, methacryl amide, alkyl- and dialkylacrylamide, alkyl- and dialkylmethacryl amide, alkyl acrylate, alkylmethacrylate, vinyl caprolactone, vinyl caprolactam, vinyl pyrrolidone, vinyl ester, e.g. vinyl acetate, vinyl alcohol, propylene glycol or ethylene glycol, wherein the alkyl groups of these monomers preferably have one to seven carbon atoms, especially one to three carbon atoms.

Suitable polymers with quaternary amine groups are, for example, polymers described in the CTFA Cosmetic Ingredient Dictionary under the name “polyquaternium”, such as methylvinylimidazolium chloride/vinyl pyrrolidone copolymer (Polyquaternium-16) or quaternized vinyl pyrrolidone/dimethylamino-ethylmthacrylate copolymer (Polyquaternium-11) and quaternary silicone polymers and/or oligomers, such as silicone polymers with quaternary terminal groups (Quaternium-80).

Preferred cationic polymers on a synthetic basis include: poly(dimethyldiallylammonium chloride); copolymers of acrylamide and dimethyldially ammonium chloride; quaternary ammonium polymers, formed by reaction of diethyl sulfate and a copolymer of vinyl pyrrolidone and dimethylaminoethylmethacrylate, especially vinyl pyrrolidone/dimethylamino-ethylmethacrylate methosulfate copolymer, (e.g. GAFQUAT® 755 N and GAFQUAT® 734); quaternary ammonium polymers of polyvinyl pyrrolidone and imidazoliminemethochloride (e.g. LUVIQUAT® HM 550); Polyquaternium-35; Polyquaternium-57; polymer from trimethyl ammonium-ethyl-methacrylate chloride; the terpolymer of dimethyldiallylammonium chloride, sodium acrylate and acrylamide (MERQUAT® Plus 3300); terpolymer of vinyl pyrrolidone, dimethylaminopropylmethacrylamide and methacryloylaminopropyllauryidimethyl ammonium chloride; terpolymer of vinyl pyrrolidone, dimethylaminoethylmethacrylate and vinyl caprolactam (e.g. GAFFIX® VC 713); vinyl pyrrolidone/methacrylamidopropyltrimethyl ammonium chloride copolymer (GAFQUAT® HS 100); copolymers of vinyl pyrrolidone and dimethylaminoethylmethacrylate; copolymers of vinyl pyrrolidone, vinyl caprolactam and dimethylaminopropyl acrylamide; polyesters or oligoesters built up from at least one first monomer type, which is a hydroxy acid substituted with at least one quaternary ammonium group; substituted dimethylpolysiloxanes with quaternary ammonium end groups.

Suitable cationic polymers, which are derived from natural polymers, are especially cationic derivatives of polysaccharides, e.g. cellulose, starch or guar. Chitosan and chitosan derivative compounds are suitable. Cationic poly-saccharides have the general formula (II):
G-O—B—N⁺R^{a}R^{b}R^{c}X⁽⁻⁾ (II),
wherein G is an anhydroglucose residue, for example starch or cellulose anhydroglucose; B is a divalent connecting group, for example, an alkylene, an oxyalkylene, a polyoxyalkylene or hydroxyalkylene;
R^{a}, R^{b} and R^{c} are each, independently of each other, alkyl, aryl, alkylaryl, arylalkyl, alkoxyalkyl or alkoxyaryl with up to 18 carbon atoms respectively, wherein the total number of carbon atoms in R^{a}, R^{b} and R^{c} is at most 20; X is a common counter anion, for example a halogen, acetate, phosphate, nirate or alkyl sulfate, preferably chloride. Preferred cationic celluloses are those marketed under the INCI name, polyquaternium-10 and polyquaternium-24. A suitable cationic guar derivative is e.g. the derivative with the INCI name, guar hydroxypropyltrimonium chloride.

Chitosan, chitosan salts and chitosan derivative compounds are especially preferred as cation-active materials. The chitosan used in the composition of the invention is partially or completely deacetylated. The molecular weights of chitosan can vary over a wide range, for example from 20,000 to 5,000,000 g/mol. A low molecular weight chitosan is, for example, considered to be a chitosan with a molecular weight of from 30,000 to 70,000 g/mol. Preferably the molecular weight of the chitosan is above 100,000 g/mol, especially preferably form 200,000 to 700,000 g/mol. The deacetylation degree amounts to from 10 to 99%, especially preferably form 60 to 99%. A preferred chitosan salt is chitosonium pyrrolidone carboxylate, which for example is marketed under the trade name KYTAMER® PC of Amerchol, USA. The chitosan obtained has a molecular weight of about 200,000 to 300,000 g/mol and is deacetylated up to 70 to 85%. Quaternary, alkylated or hydroxyalkylated derivative chitosan compounds, for example, the hydroxyethyl chitosan, hydroxypropyl chitosan or hydroxybutyl chitosan, are suitable in the compositions according to the invention. The chitosans or chitosan derivative compounds should be present in neutralized or partially neutralized form when used in the compositions of the invention. The neutralization degree for the chitosan or the chitosan derivative compounds is preferably at least 50%, especially preferably between 70 and 100%, relative to the number of free base groups. In principle, all cosmetically compatible inorganic or organic acids may be used as neutralization agent, for example formic acid, tartaric acid, malic acid, lactic acid, citric acid, pyrrolidone carboxylic acid, hydrochloric acid, among other. Pyrrolidone carboxylic acid is especially preferred as neutralization agent.

Preferred cationic polymers on a natural basis include: cationic cellulose derivatives comprising hydroxyethyl cellulose and diallyldimethyl ammonium chloride; cationic cellulose derivates comprising hydroxyethyl cellulose and with trimethyl ammonium substituted epoxide; chitosan and its salts; hydroxyalkyl chitosans and their salts; alkylhydroxyalkyl chitosans and their salts; N-hydroxyalkyl chitosan alkyl ether; N-hydroxyalkylchitosan benzyl ether.

In other embodiments of the invention the composition can contain from 0.01 to 15 percent by weight, preferably from 0.5 to 10 percent by weight, of at least one synthetic or natural nonionic film-forming polymer. The term “natural polymer” includes polymers of natural origin that are chemically modified. The term “film-forming polymer” means those polymers, which deposit a polymer on the hair from 0.01 to 5% aqueous, alcoholic or aqueous-alcoholic solution, when the solution is applied to the hair.

Suitable synthetic nonionic polymers include homopolymers or copolymers, which are built up from at least one of the following monomers: vinyl pyrrolidone, vinyl caprolactam, vinyl esters, such as vinyl acetate, vinyl alcohol, acrylamide, methacrylamide, alkyl- and dialkylacrylamide, alkyl- and dialkylmethacrylamide, alkylacrylate, alkylmethacrylate, propylene glycol or ethylene glycol, wherein the alkyl groups in these monomers preferably have from one to seven carbon atoms, especially preferably from one to three carbon atoms. For example, homopolymers of vinyl caprolactam, of vinyl pyrrolidone or of N-vinylformamide, are especially suitable. Additional suitable synthetic film-forming nonionic hair-fixing polymers are, e.g., copolymerizates of vinyl pyrrolidone and vinyl acetate, terpolymers of vinyl pyrrolidone, vinyl acetate and vinyl propionate, polyacrylamide, polyvinyl alcohols, and polyethylene glycol/polypropylene glycol copolymers. Suitable natural film-forming polymers include e.g. cellulose derivatives, e.g. hydroxyalkyl cellulose.

Preferred nonionic polymers include: polyvinyl pyrrolidone, polyvinyl caprolactam, vinyl pyrrolidone/vinyl acetate copolymers, polyvinyl alcohol, isobutylene/ethylmaleimide/hydroxyethyl maleimide copolymer; copolymers of vinyl pyrrolidone, vinyl acetate and vinyl propionate.

In various embodiments of the invention the composition according to the invention can contain at least one silicone compound in an amount of preferably 0.01 to 15 percent by weight, especially preferably from 0.1 to 5 percent by weight, as hair-care ingredient. The silicone compounds include volatile and non-volatile silicones and soluble and insoluble silicones. In one embodiment the silicone compounds are high molecular weight silicones with a viscosity of 1,000 to 2,000,000 cSt at 25° C., preferably 10,000 to 1,800,000 or 100,000 to 1,500,000. The silicone compounds include polyalkyl- and polyarysiloxanes, especially with methyl-, ethyl-, propyl-, phenyl-, methylphenyl- and phenylmethyl groups. Polydimethylsiloxanes, polydiethylsiloxanes, polymethylphenyl siloxanes are preferred. Luster-imparting, arylated silicones with an index of refraction of at least 1.46 or at least 1.52 are particularly preferred. The silicone compounds especially include materials with the INCI names: cyclomethicone, dimethicone, dimethiconol, dimethicone copolyol, phenyl trimethicone, amodimethicone, trimethylsilylamodimethicone, stearyl siloxysilicates, polymethylsilsesquioxane and dimethicone cross polymers. Silicone resins and silicone elastomers, which are highly cross-linked siloxanes, are also suitable.

Preferred silicones are: cyclic dimethylsiloxanes, linear polydimethylsiloxanes, block polymers comprising polydimethyl siloxanes and polyethylene oxides and/or polypropylene oxides, polydimethyl siloxanes with terminal or side polyethylene oxide or polypropylene oxide groups, polydimethyl siloxanes with terminal hydroxy groups, phenyl substituted polydimethyl siloxanes, silicone emulsions, silicone elastomers, silicone waxes, silicone gums and amino-substituted silicones.

In other embodiments the composition according to the invention contains preferably from 0.01 to 10 percent by weight or from 0.1 to 5 percent by weight, especially preferably from 0.2 to 2 percent by weight, of a light-protective substance as hair protective ingredient. The light-protective substance especially include especially are all those disclosed in EP 1 084 696. The following light-protective substances are especially preferred: 4-methoxycinamic acid-2-ethylhexyl ester, methylmethoxy cinnamate, 2-hydroxy-4-methoxybenzophenon-5-sulfonic acid and polyethoxylated p-aminobenzoate.

Various embodiments of the composition according to the invention also contain at least one hydrophobic oil or wax in an amount of preferably from 0.01 to 20 or from 0.05 to 10, especially preferably from 0.1 to 5, percent by weight, as a hair-care or hair-fixing effective ingredient.

The liquid hydrophobic oil has a melting point of less than or equal to 25° C. and a boiling point of preferably over 250° C., especially over 300° C. In principle all oils known to those skilled in the art can be used for this purpose. These oils include plant oils, animal oils, mineral oils (paraffin liquidum), silicone oils or their mixtures. Hydrocarbon oils, e.g. paraffin oils, isoparaffin oils, squalane, oils from fatty acids and polyols, especially triglyceides, are suitable. In addition plant oils, such as sunflower seed oil, coconut oil, castor oil, lanolin oil, jojoba oil, corn oil and soybean oil, are suitable.

In principle any wax known to those skilled in the art can be used as the wax or waxy material. The following are suitable for this purpose: animal waxes, plant waxes, mineral waxes and synthetic waxes, microcrystalline waxes, macrocrystalline waxes, solid paraffin waxes, petrolatum, VASELINE®, osocerite, montan wax, Fischer-Tropsch wax, polyolefin waxes, for example polybutylene, beeswax, wool wax and its derivatives, such as wool wax alcohol, candelilla wax, olive wax, carnauba wax, Japan wax, apple wax, hardened fats, fatty acid esters and fatty acid glycerides each with a hardening point of above 40° C., polyethylene glycol waxes and silicone waxes. The waxes or waxy materials have a solidification point above 40° C., preferably above 55° C. The needle penetration number (0.1 mm, 100 g, 5 s, 25° C.; according to DIN 51 579) preferably is in a range from 2 to 70, especially from 3 to 40.

Various other embodiments of the composition according to the invention contain from 0.01 to 20, preferably from 0.05 to 10, and especially preferably from 0.1 to 5, percent by weight of at least one hair conditioning additive ingredient selected from betaine, panthenol, panthenyl ethyl ether, sorbitol, protein hydroylyzates, plant extracts, A-B block copolymers of alkyl acrylates and alkylmethacrylates, A-B block copolymers of alkylmethacrylates and acryl nitrile, A-B-A block copolymers from lactide and ethylene oxide, A-B-A block copolymers of caprolactone and ethylene oxide, A-B-C block copolymers of alkylene or alkadiene compounds, styrene and alkylmethacrylates, A-B-C block copolymers of acrylic acid, styrene and alkylmethacrylates, star-form block copolymers, hyperbranched polymers, dendrimers, intrinsically electrically conductive 3,4-polyethylenedioxythiophenes and intrinsically electrically conductive polyanilines.

Many different embodiments of the composition according to the invention contain from 0.01 to 20, preferably from 0.05 to 10, and especially preferably from 0.1 to 5, percent by weight of at least one hair-care and/or hair-cleansing surfactant, as hair-care and/or hair-cleansing effective ingredient. The surfactants can be nonionic, anionic, cationic or zwitterionic.

Suitable nonionic surfactants include, e.g.:
ethoxylated fatty alcohols, fatty acids, fatty acid glycerides or alkylphenols, especially addition products of 2 to 30 mol ethylene oxide and/or 1 to 5 mol propylene oxide to C₈- to C₂₂-fatty alcohols, addition products of 2 to 30 mol ethylene oxide and/or 1 to 5 mol propylene oxide to C₁₂- to C₂₂-fatty acids, addition products of 2 to 30 mol ethylene oxide and/or 1 to 5 mol propylene oxide to alkylphenols with 8 to 15 carbon atoms in their alkyl groups;
C₁₂- to C₂₂-fatty acid mono- and diesters of addition products of 1 to 30 mol ethylene oxide with glycerol;
addition products of 5 to 60 mol of ethylene oxide with castor oil or hardened (hydrogenated) castor oil;
fatty acid sugar esters, especially esters of sucrose with one or two fatty acids with 8 to 22 carbon atoms, such as sugar cocoate, sucrose dilaurate, sucrose distearate, sucrose laurate, sucrose myristate, sucrose oleate, sucrose palmitate, sucrose ricinoleate, sucrose stearate;
esters of sorbitan with one, two or three fatty acids with 8 to 22 carbon atoms and with an ethoxylation degree of 4 to 20;
polyglyceryl fatty acid esters, especially of one, two or several fatty acids with 8 to 22 carbon atoms and polyglycerol with preferably 2 to 20 glyceryl units; and
alkylglucosides, alkyloligoglucosides and alkyl polyglucosides with 8 to 22 alkyl groups, e.g. decyl glucoside or lauryl glucoside.

Suitable anionic surfactants are, e.g. salts and esters of carboxylic acids, alky ether sulfates and alkyl sulfates, fatty alcohol ether sulfates, sulfonic acids and their salts (e.g. sulfosuccinate or fatty acid isethienate), phosphoric acid ester and their salts, acylamino acids and their salts. These anionic surfactants are described in the publication “FIEDLER—Lexicon of Auxiliary Substances” Volume 1, fifth edition (2002), pp. 97 to 102. Preferred surfactants are monoesters, diesters or triesters of phosphoric acid with addition products of 2 to 30 mol of ethylene oxide with C₈- to C₂₂-fatty alcohols.

Suitable amphoteric surfactants are derivatives of aliphatic quaternary ammonium-, phosphonium- and sulfonium compounds of the general formula (III):
R¹—Y⁽⁺⁾(R²)ₙCH₂—R³-Z⁽⁻⁾ (III),
wherein R¹ denotes a linear or branched alkyl-, alkenyl- or hydroxyalkyl group with 8 to 18 carbon atoms and 0 to about 10 ethylene oxide units and 0 to 1 glyceryl units; Y denotes an N-, P-, or S-containing group; R² is an alkyl or mono-hydroxyalkyl group with 1 to 3 carbon atoms; x is 1, in case Y is an S atom and x is 2 when Y is an N or a P atom; R³ denotes an alkylene or hydroxyalkyl group with 1 to 4 carbon atoms and Z denotes a carboxylate, sulfate, phosphonate or phosphate group.

Additional suitable amphoteric surfactants are those, which are derived from betaine, e.g. C₈- to C₁₈-alkylbetaines, such as cocodimethylcarboxymethylbetaine, lauryldimethylcarboxymethylbetaine, lauryldimethyl-alpha-carboxyethylbetaine, cetyldimethylcarboxymethylbetaine, oleyldimethyl-gamma-carboxypropylbetaine or lauryl-bis-(2-hydroxypropyl)-alpha-carboxyethylbetaine; C₈- to C₁₈-alkylsulfobetaines, such as cocodimethylsulfopropylbetaine, stearyl-dimethyl-sulfopropylbetaine, lauryldimethylsulfoethylbetaine, lauryl-bis-(2-hydroxyethyl)-sulfopropylbetaine; the carboxyl derivatives of imidazole, the C₈- to C₁₈-alkyldimethylammonium acetates, the C₈- to C₁₈-alkyldimethyl-carbonylmethylammonium salts and the C₈- to C₁₈-fatty acid alkylamidobetaines, such as coconut fatty acid amidopropylbetaine, and the N-coconut fatty acid amidoethyl-N-[2-carboxymethoxy)-ethyl]glycinate (CTFA name cocoamphocarboxyglycinate).

Suitable cationic surfactants contain amino groups or quaternarized hydrophilic ammonium groups, which carry a positive charge in solution and s can be represented by the following general formula (IV):
N⁺R¹R²R³R⁴X⁽⁻⁾ (IV),
wherein each of R¹ to R⁴, independently of each other, denote an aliphatic group, an aromatic group, an alkoxy group, a polyoxyalkylene group, an alkylamido group, a hydroxyalkyl group, an aryl group or an alkaryl group, each with from 1 to 22 carbon atoms, wherein each of the R¹ to R⁴ groups has at least six, preferably at least eight carbon atoms. X⁽⁻⁾ is a cosmetically compatible anion. The anion can be a halogen, acetate, phosphate, nitrate or alkyl sulfate anion. However chloride is preferred. The aliphatic groups can also contain cross-linkages or other groups, for example amino groups, besides the carbon atoms and the hydrogen atoms. For example, suitable cationic surfactants include the chlorides or bromides of alkyldimethylbenzylammonium salts, alkyltrimethylammonium salts, e.g. cetyltrimethylammonium chloride or bromide, tetradecyltrimethylammonium chloride or bromide, alkyldimethylhydroxyethylammonium chloride or bromide, dialkyldimethylammonium chloride or bromide, alkylpyridinium salts, especially lauryl or cetyl pyridinium chloride, alkylamidoethyltrimethylammonium ether sulfate and compounds with cationic character, such as amine oxides, especially alkylmethylamine oxides or alkylaminoethyldimethylamine oxides. C₈- to C₂₂-alkyldimethylbenzylammonium compounds, C₈- to C₂₂-alkyltrimethylammonium compounds, especially cetyltrimethylammonium chloride, C₈- to C₂₂-alkyldimethylhydoxyethylammonium compounds, di-(C₈- to C₂₂-alkyl)-dimethylammonium compounds, C₈- to C₂₂-alkylpyridinium salts, C₈- to C₂₂-alkylamidoethyltrimethylammonium ether sulfate, C₈- to C₂₂-alkylmethylamide oxide and C₈- to C₂₂-alkylaminoethyldimethylamine oxide.

A special embodiment of the invention concerns a hair care agent. The hair care agent is, for example, a conditioner, a treatment, a hair care, rinse or the like. The hair care agent contains at least one hair care effective ingredient, which is selected from the above-described silicone compounds, cationic surfactants, amine substituted surfactants, cationic polymers and amine substituted polymers. The hair care effective ingredient can be contained in the composition of the invention in an amount of between 0.01 to 10.0 percent by weight, especially between 0.01 and 5.0 percent by weight, based on a total amount of finished product. The hair care composition according to the invention be left on the hair or rinsed from the hair after a suitable acting time. The acting times depend on the type of hair. As a general guideline acting times of between 0.5 and 30 minutes, especially from 0.5 and 10 minutes, preferably between 1 to 5 minutes.

Besides the above-described cationic surfactants additional suitable cationic or amine-substituted surfactants are those of the formula R1-NH—(CH₂)ₙ—NR2R3 or of the formula R1-NH—(CH₂)ₙ—N⁺R2R3R4 X⁻, wherein R1 denotes an acyl or alkyl group with 8 to 24 carbon atoms, which is branched or unbranched, saturated or unsaturated, wherein the acyl and/or the alkyl group can contain one or more OH groups; wherein R2, R3 and R4, independently of each other, are hydrogen, alkyl or alkoxyalkyl groups with 1 to 6 carbon atoms, which are the same or different saturated or unsaturated and can be substituted with one or more hydroxyl groups; X⁻ is an anion, especially a halide ion, and a compound of the general formula RSO₃⁻, wherein R denotes a saturated or unsaturated aliphatic group with 1 to 4 carbon atoms, and n denotes a whole number between 1 and 10, preferably from 2 to 5.

Preferably the hair care ingredient is an amidoamine and/or a quaternary amidoamine of the above-described formula, wherein R1 is a branched or unbranched saturated or unsaturated acyl group with 8 to 24 carbon atoms, which can contain at least one OH group. Those amines and/or quaternarized amines, in which at least one of the groups R2, R3 and R4 denote a group of the formula CH₂CH₂OR5, wherein R5 can be an alkyl group with 1 to 4 carbon atoms, hydroxyethyl or H. Suitable amines or amidoamines, which can be quaternized, are e.g. those with the INCI name ricinolamidopropyl betaine, ricinolamidopropyl dimethylamine, ricinolamidopropyl dimethyl lactate, ricinolamidopropyl ethyldimonium ethosulfate, ricinolamidopropyltrimonium chloride, ricinolamidopropyltrimonium methosulfate, cocamidopropyl betaine, cocamidopropyl dimethylamine, cocamidopropyl ethyldimonium ethosulfate, cocamidopropyltrimonium chloride, behenamidopropyl dimethylamine, isostearylamidopropyl dimethylamine, stearylamidopropyl dimethylamine, Quaternium-33, undecyleneamidopropyltrimonium methosulfate.

In various other embodiments the composition according to the invention can contain at least one hair-coloring pigment, which imparts color and/or a luster effect to the hair. The coloring or luster effects on the hair are preferably temporary, i.e. they last until the next hair washing and can be removed again from the hair by washing the hair with conventional shampoos. The pigments are present in the product mass in undissolved form and can be contained in an amount of 0.01 to 25 wt. %, especially preferably from 5 to 15 wt. %. The preferred particle size amounts to from 1 to 200 μm, especially from 3 to 150 μm, especially preferably from 10 to 100 μm. The pigments are coloring agents that are practically insoluble in application media and can be organic or inorganic. Also inorganic-organic mixed pigments are possible. Inorganic pigments are preferred. The advantage of the inorganic pigments is their outstanding light-resistance, weather-resistance and temperature-resistance. The inorganic pigments can be of natural origin, for example chalk, ocher, umber, green earth, burnt siena or graphite. The pigments can be white pigments, such as titanium dioxide or zinc oxide; black pigments, such as iron oxide black; fancy or multi-colored pigments, such as ultramarine or iron oxide red; lustrous pigments, metal effect pigments, pearlescent pigments as well as fluorescene or phosphorescent pigments. Preferably at least one pigment is a colored, non-white pigment. Metal oxides, metal hydroxides and metal oxide hydrates, mixed phase pigments, sulfur-containing silicates, metal sulfides, complex metallo-cyanides, metal sulfates, metal chromates and metal molybdates and metals themselves (bronze pigments) are suitable. In particular, titanium dioxide (C.I. 77891), black iron oxide (C.I. 77499), yellow iron oxide (C.I. 77492), red and brown iron oxide (C.I. 77491), manganese violet (C.I. 77742), ultramarine (sodium aluminum sulfosilicate, C.I. 77007, Pigment Blue 29), chromium oxide hydrate (C.I. 77289), Iron Blue (Ferric ferrocyanide, C.I. 77510) and carmine (cochineal), are all suitable pigments.

Pigments based on mica and/or isinglass, which are coated with a metal oxide or metal oxychloride, such as titanium dioxide or bismuth oxychloride and if necessary other color-imparting materials, such as iron oxides, iron blue, ultramarine, carmine, etc, whose colors can be modified by changing the thickness of the coating, are especially preferred. Pigments of this sort are marketed, for example, under the trademark, Rona®, Colorona®, Dichrona® and Timiron® by the firm, Merck, Germany.

Organic pigments are, for example, the natural pigments, Sepia, gamboge, charcoal, Kasseler brown, indigo, chlorophyl and other plant pigments. Synthetic organic pigments include, for example, azo pigments, anthrquinoid pigments, indigoid pigments, dioxazine, quinacridone, phthalocyanine isoindolinone pigments, perylene pigments, perinone pigments, metal complex pigments, alkali blue pigments and diketopyrrolopyrrole pigments.

Additional embodiments of the composition according to the invention are agents for permanent shaping of hair. These compositions contain a reducing agent, especially a keratin-reducing mercapto compound, in an amount of preferably from 0.5 to 15 percent by weight. The permanent wave composition preferably is an aqueous alkaline (pH=5 to 10) preparation, which contains cysteine, cysteamine, N-acetyl-L-cysteine, mercapto carboxylic acids, such as e.g. thioglycolic acid or thiolactic acid, or salts of mercapto carboxylic acids, such as ammonium and guanidine salts of thioglycolic acids or thiolactic acids. The required alkalinity is adjusted by addition of ammonia, organic amines, ammonium carbonate, alkali carbonates or hydrogen carbonates. However the composition can be a neutral or acidic (pH=4.5 to 7) hair shaping composition, which contains sulfites or mercapto carboxylic acid esters in aqueous media. In the first case preferably sodium or ammonium sulfites or salts of sulfuric acid with an organic amine, such as monoethanolamie and guanidine, are used in a concentration of about 1 to 12 wt. % (calculated as SO₂). In the latter case thioglycolic acid monoglycolic ester or glyceryl ester is used in a concentration of about 5 to 50% by weight (corresponding to a content of free thioglycolic acid of 2 to 16% by weight). The composition according to the invention for permanent hair shaping can contain a mixture of the above-described keratin-reducing compounds. After an acting time sufficient for the permanent shaping of the hair the hair is rinsed with water and subsequently oxidatively after-treated (fixed). A fixing agent containing at least one oxidizing agent or any fixing agent used currently for this sort of treatment can be used for the oxidative after-treatment. For example, sodium and potassium bromate, sodium perborate, urea peroxide and hydrogen peroxide can be used as the oxidizing agent in the sort of fixing agent. The concentration of the oxidizing agent can be from about 0.5 to 10 percent by weight, so that the fixing agent according to the invention can preferably contain from 0.05 to 5 percent by weight of the spherical particles. Both the composition for permanent hair shaping according to the invention and the fixing agent according to the invention can be in the form of an aqueous solution or emulsion and in thickened form on an aqueous basis for example as a cream, gel or paste. Furthermore it is also possible to fill these compositions under pressure in an aerosol can or container and later dispense them as foam. The method for permanent shaping described above permits a careful and uniform shaping from the hair tips to the hair roots, provides outstanding wet and dry combability, a pleasant feel and luster in the dry state and loose, springy and uniform permanent shaping, especially in the vicinity of the hair tips.

Other embodiments of the composition according to the invention are hair dye agents or hair tinting or coloring agents. They can be in the form of an oxidative hair dye composition comprising oxidative dyestuffs or a non-oxidative hair dye composition including direct-dyeing dye compounds. The total amount of the oxidation dye precursor compounds containing in these embodiments of the composition according to the invention is preferably from 0.01 to 12 percent by weight, especially from 0.2 to 6 percent by weight. For example the following developer substances and coupler substances and self-coupling compounds can be oxidative dye precursor compounds used in the oxidation dye compositions according to the invention.

Developers:

1,4-Diaminobenzene (p-Phenylendiamine), 1,4-Diamino-2methylbenzene (p-toluylene diamine), 1,4-Diamino-2-(thiophen-2-yl)-benzene, 1,4-Diamino-2-(thiophen-3-yl)benzene, 1,4-Diamino-2-(pyridin-3-yl)benzene, 2, 5-Diaminobiphenyl, 1,4-Diamino-2-methoxymethyl-benzene, 1,4-Diamino-2-aminomethylbenzene, 1,4-Diamino-2-hydroxymethyl-benzene, 4-[Di-(2-hydroxyethyl)amino]-aniline, 1,4-Diamino-2-(1-hydroxyethyl)-benzene, 1,4-Diamino-2-(2-hydroxyethyl)-benzene, 1,3-Bis-[(4-aminophenyl)-(2-hydroxyethyl) amino]-2-propanol, 1,8-Bis (2,5-diaminophenoxy)-3,6-dioxaoctane, 2,5-Diamino-4′-Hydroxy-1,1′-biphenyl, 2,5-Diamino-2′-trifluoromethyl-1,1′-biphenyl, 2,4′,5-Triamino-1,1′-biphenyl, 4-Aminophenol, 4-Amino-3-methylphenol, 4-Methylamino-phenol, 4-Amino-2-(aminomethyl)-phenol, 4-Amino-2-[(2-hydroxyethyl)-amino]methyl-phenol, 4-Amino-2-(methoxymethyl)-phenol, 5-Aminosalicylic acid, 2,4,5,6-tetramino-pyrimidine, 2,5,6-Triamino-4-(1H)-pyrimidone, 4,5-Diamino-1-(2-hydroxyethyl)-1H-pyrazole, 4,5-Diamino-1-pentyl-1H-pyrazole, 4,5-Diamino-1-(phenylmethyl)-1H-pyrazole, 4,5-Diamino-1-((4-methoxyphenyl)methyl-1H-pyrazole, 2-Aminophenol, 2-Amino-6-methylphenol, 2-Amino-5-methyl-phenol, 1,2,4-Trihydroxy-benzene, 2,4-Diaminophenol, 1,4-Dihydroxybenzene, 2-(((4-Aminophenyl)-amino)methyl)-1,4-diaminobenzene.

Couplers:

N-(3-Dimethylaminophenyl)-urea, 2,6-Diamino-pyridifi, 2-Amino-4-[(2-hydroxyethyl)-amino]-anisole, 2,4-Diamino-1-fluoro-5-methyl-benzene, 2,4-Diamino-1-ethoxy-5-methylbenzene, 2,4-Diamino-1-(2-hydroxyethoxy)-5-methylbenzene, 3-Amino-6-methoxy-2-(methylamino)-pyridine, 3,5-Diamino-2,6-dimethoxypyridine, 1,3-Diaminobenzene, 2,4-Diamino-1-(2-hydroxyethoxy)-benzene, 1,3-Diamino-4-(3-hydroxypropoxy)benzene, 1,3-Diamino-4-(2-methoxyethoxy)-benzene, 1,3-Di-(2,4-diaminophenoxy)-propane, 2,6-Bis-(2-hydroxyethyl)-aminotoluene, 5-Amino-2-methylphenol, 5-Amino-4-fluoro-2-methylphenol, 3-Amino-2,4-dichlorophenol, 3-Amino-2-chloro-6-methylphenol, 3-Aminophenol, 5-[(2-Hydroxyethyl)-amino]-2-methylphenol, 2-Amino-3-hydroxypyridine, 2,6-Dihydroxy-3,4-dimethylpyridine, 5-Amino-4-chloro-2-methylphenol, 1-Naphthol, 1,5-Dihydroxynaphthalene, 1,7-Dihydroxy-naphthalene, 2,7-Dihydroxynaphthalene, 2-Methyl-1-naphthol acetate, 1,3-Dihydroxybenzene, 1-Chloro-2,4-dihydroxybenzene, 1,3-Dihydroxy-2-methylbenzene-5-[(2-Hydroxyethyl)amino]-1,3-benzodioxole, 3,4-Diamino-benzoic acid, 3,4-Dihydro-6-hydroxy-1,4(2H)-benzoxazine, 3-Methyl-1-phenyl-5-pyrazolone, 5,6-Dihydroxyindole, 5,6-Dihydroxy-indolene, 6-Hydroxyindole, 2,3-Indolindione.

The total amount of direct-dyeing dye compound in the composition according to the invention amounts to about 0.01 to 7 percent by weight, preferably about 0.2 to 4 percent by weight. Suitable direct-dyeing dye compounds, e.g., are triphenylmethane dye compounds, aromatic nitro dye compounds, azo dye compound, quinone dye compounds and cationic or anionic dye compounds. The following dye compounds are especially preferred.

Nitro Dye Compounds (Blue):

1,4-Bis-[(2-hydroxyethyl)amino]-2-nitrobenzene, 1-(2-Hydroxyethyl) amino-2-nitro-4-[di-(2-hydroxyethyl)amino]-benzene, (HC Blue No. 2), 1-Amino-3-methyl-4-[(2-Hydroxy-ethyl)amino]-6-nitrobenzene, (HC Violet No. 1), 4-[Ethyl-(2-hydroxyethyl) amino]-1-[(2-hydroxyethyl)-amino]-2-nitrobenzene hydrochloride (HC Blue No. 12), 1-[(2,3-Dihydroxypropyl)amino]-4-[methyl-(2-hydroxyethyl)-amino]-2-nitrobenzene (HC Blue No. 10), 1-[(2,3-Dihydroxypropyl)amino]-4-[ethyl-(2-hydroxyethyl)amino]-2-nitrobenzene-hydrochloride (HC Blue No. 9), 1-(3-Hydroxypropylamino)-4-[di-(2-hydroxyethyl)-amino]-2-nitrobenzene, (HC Violet No. 2)

Nitro Dye Compounds (Red):

1-Amino-4-[(2-hydroxyethyl)amino]-2-nitrobenzene (HC Red No. 7), 2-Amino-4,6-dinitrophenol, 1,4-Diamino-2-nitrobenzene (CI 76070) (HC Red No. 1), 1-Amino-4-[di-(2-hydroxyethyl)-amino] (HC Red No. 13), 1-Amino-5-chloro-4-[(2-hydroxyethyl)amino]-2-nitrobenzene, 4-Amino-1-[(2-hydroxyethyl)amino]-2-nitrobenzene (HC Red No. 3), 4-Amino-2-nitro-1-((prop-2-en-1-yl)-amino)-benzene, 4-Amino-3-nitrophenol, 4-[(2-Hydroxyethyl)-amino]-3-nitrophenol, 4-[(2-Nitrophenyl)-amino]phenol (HC Orange No. 1), 1-[(2-Aminoethyl)-amino]-4-(2-hydroxyethoxy)-2-nitrobenzene (HC Orange No. 2), 4-(2,3-Dihydroxypropoxy)-1-[(2-hydroxyethyl)-amino]-2-nitrobenzene (HC Orange No. 3), 1-Amino-5-Chloro-4-[(2,3-dihydroxy-propyl)-amino]-2-nitrobenzene (HC Red No. 10), 5-Chloro-1,4-[di-(2,3-dihydroxy-propyl)amino]-2-nitrobenzene (HC Red No. 11), 2-[(2-Hydroxyethyl)amino]-4,6-dinitrophenol, 4-Ethylamino-3-nitrobenzoic acid, 2-[(4-Amino-2-nitrophenyl)-amino]benzoic acid, 2-Chloro-6-ethylamino-4-nitrophenol, 2-Amino-6-chloro-4nitrophenol, 4-[(3-Hydroxypropyl)amino]-3-nitrophenol, 2,5-Diamino-6-nitropyridine, 3-Amino-6-(methylamino)-2-nitropyridine, 1,2,3,4-Tetrahydro-6-nitroquinoxaline, 7-Amino-3,4-dihydro-6-nitro-2H-1,4-benzoxazine (HC Red No. 14).

Nitro Dye Compounds (Yellow):

1,2-Diamino-4-nitrobenzene (CI 76020), 1-Amino-2-[(2-hydroxy-ethyl)amino]-5-nitrobenzene (HC Yellow No. 5)), 1-(2-Hydroxyethoxy)-2-1 (2-hydroxyethyl)-amino]-5-nitrobenzene, (HC Yellow No. 4), 1-[(2-Hydroxyethyl)amino]-2-nitrobenzene (HC Yellow No. 2), 2-((2-Hydroxyethyl)amino]-1-methoxy-5-nitrobenzene, 2-Amino-3-nitrophenol, 1-Amino-2-methyl-6-nitrobenzene, 1-(2-hydroxyethoxy)-3-methylamino-4-nitrobenzene, 2,3-(Dihydroxypropoxy)-3-methylamino-4-nitrobenzene, 2-[(2-Hydroxy-ethyl)-amino]-5-nitrophenol (HC Yellow No. 11), 3-[(2-Aminoethyl)-amino]-1-methoxy-4-nitrobenzene hydrochloride (HC Yellow. No. 9), 1-[(2-Ureidoethyl)amino]-4-nitrobenzene, 4-[(2,3-Dihydroxypropyl)amino]-3-nitro-1-trifluormethylbenzene, (HC Yellow No. 6), 1-Chloro-2,4-bis[(2-hydroxyethyl)amino]-5-nitrobenzene (HC Yellow No. 10), 4-[(2-Hydroxyethyl)amino]-3-nitro-1-methylbenzene, 1-Chloro-4-[(2-hydroxyethyl)-amino]-3-nitrobenzene (HC Yellow No. 12), 4-[(2-Hydroxyethyl)amino]-3-nitro-1-trifluormethyl-benzene (HC Yellow No. 13), 4-[(2-Hydroxyethyl)amino]-3-nitro-benzonitrile (HC Yellow No. 14), 4-((2-Hydroxyethyl)amino]-3-nitro-benzamide (HC Yellow No. 15), 2,4-Dinitro-1-hydroxynaphthalene.

Quinone Dye Compounds:

1,4-Di-[(2,3-dihydroxypropyl)amino]-9,10-anthraquinone, 1,4-Di-[(2-hydroxyethyl)amino]-9,10-anthraquinone (CI 61545, Disperse Blue 23), 1-Amino-4-hydroxy-9,10-anthraquinone (CI 60710, Disperse Red 15), 1-Hydroxy-4-[(4-methyl-2-sulfophenyl)amino]-9,10-anthraquinone, 7-Beta-D-glucopyranosyl-9,10-dihydro-1-methyl-9,10-dioxo-3,5,6,8-tetrahydroxy-2-anthracene carboxylic acid (CI 75470, Natural Red 4),1-[(3-Aminopropyl)amino]-9,10-anthraquinone (HC Red No. 8), 1,4-Diamino-9,10-anthraquinone (CI 61100, Disperse Violet No.2), 1-Amino-4-(methylamino)-9,10-anthraquinone (CI 61105, Disperse Violet No. 4, Solvent Violet No. 12), N-(6-((3-Chloro-4-(methylamino)phenyl)imino)-4-methyl-3-oxo-1,4-cyclohexadien-1-yl)-urea (HC Red No. 9), 2-((4-(Di(2-hydroxyethyl)-amino)phenyl)amino)-5-((2-hydroxyethyl)amino)-2,5-cyclohexadien-1,4-dione (HC Green No. 1), 2-Hydroxy-1,4-naphthoquinone (CI 75480, Natural Orange No. 6), 1,2-Dihydro-2-(1,3-dihydro-3-oxo-2H-indol-2-ylidene)-3H-indol-3-one (CI 73000), 1,3-Bis(dicyanomethylen)indane.

Basic Dye Compounds:

Di-[4-(diethylamino)phenyl][4-(ethylamino)-naphthyl]-carbenium chloride (CI 42595, Basic Blue No. 7), Di-[4-(dimethylamino)-phenyl][4-(phenylamino)-naphthyl]-carbenium chloride (CI 44045, Basic Blue No. 26), Basic Blue No. 77, 8-Amino-2-bromo-5-hydroxy-4-imino-6-[(3-(trimethylammonio) phenyl)amino]-1-(4H)-naphthalenone chloride (CI 56059, Basic Blue No. 99), Tri(4-amino-3-methylphenyl)carbenium chloride (CI 42520, Basic Violet No. 2), Di-(4-amino-phenyl) (4-amino-3-methylphenyl)-carbenium chloride (CI 42510, Basic Violet No. 14), 1-[(4-Aminophenyl)azo]-7-(trimethylammonio)-2-naphthol chloride (CI 12250, Basic Brown No. 16), 3-[(4-Amino-2,5-dimethoxyphenyl)-azo]-N,N,N-trimethyl-benzeneaminium chloride (CI 112605, Basic Orange No. 69), 1-[(4-Amino-2-nitrophenyl)-azo]-7-(trimethylammonio)-2-naphthol chloride (Basic Brown No. 17), 1-[(4-Amino-3-nitrophenyl)azo]-7-(trimethylammonio)-2-naphthol chloride (CI 12251, Basic Brown No. 17), 2-((4-Aminophenyl)azo)-1,3-dimethyl-1H-imidazol-3-ium chloride (Basic Orange No. 31), 3,7-Diamino-2,8-dimethyl-5-phenyl-phenazinium chloride (CI 50240, Basic Red No. 2), 1,4-Dimethyl-5-[(4-(dimethylamino)phenyl)azo]-1,2,4-triazolium chloride (CI 11055, Basic Red No. 22), 1,3-Dimethyl-2-((4-dimethylamino)phenyl)azo-1H-imidazol-3-ium chloride (Basic Red No. 51), 2-Hydroxy-1-[(2-methoxyphenyl)azo]-7-(trimethylammonjo)-naphthalene chloride (CI 12245, Basic Red No. 76), 3-Methyl-1-phenyl-4-[(3-(trimethylammonio)phenyl)azo]-pyrazole-5-one chloride (CI 12719, Basic Yellow No. 57), 1-Methyl-4-((methylphenylhydrazono)-methyl)-pyridinium methylsulfate (Basic Yellow No. 87), 1-(2-Morpholinium-propylamino)-4-hydroxy-9,10-anthraquinone methylsulfate, 1-[(3-(Dimethyl-propylaminium)propyl)amino]-4-(methylamino)-9,10-anthraquinone chloride.

Neutral Azo Dye Compounds:

1-[Di-(2-hydroxyethyl)amino]-3-methyl-4-[(4-nitrophenyl)azo]-benzene (CI 11210, Disperse Red No. 17), 1-[Di(2-hydroxyethyl)amino]-4-[(4-nitrophenyl)azo]-benzene (Disperse Black No. 9), 4-[(4-Aminophenyl)azo]-1-[di(2-hydroxyethyl)-amino]-3-methylbenzene (HC Yellow No. 7), 2,6-Diamino-3-[(pyridin-3-yl)azo]-pyridine, 2-((4-(Acetylamino)phenyl)azo)-4-methylphenol (CI 11855, Disperse Yellow No. 3), Disperse Blue No. 106 (CI 111935).

Acid Dye Compounds:

6-Hydroxy-5-[(4-sulfophenyl)azo]-2-naphthalene sulfonic acid disodium salt (CI 15985, Food Yellow No. 3, FD&C Yellow No. 6), 2,4-Dinitro-1-naphthol-7-sulfonic acid disodium salt (CI 10316, Acid Yellow No. 1, Food Yellow No. 1), 2-(Indan-1,3-dion-2-yl) quinoline-x,x-sulfonic acid (mixture of mono and disulfonic acids) (CI 47005, D&C Yellow No. 10, Food Yellow No. 13, Acid Yellow No. 3), 5-Hydroxy-1-(4-sulfophenyl)-4-[(4-sulfophenyl)-azo]pyrazole-3-carboxylic acid trisodium salt (CI 19140, Food Yellow No. 4, Acid Yellow No. 23), 5-[(2,4-Dinitro-phenyl)amino]-2-phenylamino-benzene sulfonic acid sodium salt (CI 10385, Acid Orange No. 3), 4-[(2-Hydroxynaphth-1-yl)azo]-benzene sulfonic acid sodium salt (CI 15510, Acid Orange No. 7), 4-((2-Hydroxynaphthalene-1-yl)azo)-3-methyl-benzene sulfonic acid sodium salt (CI 15575, Acid Orange No. 8), 3′,6′-Dihydroxy-4′,5′-diiodospiro(isobenzofuran-1 (3H)-9′-(9H)xanthen)-3-one (CI 45425, D&C Orange No. 10), 4-Hydroxy-3-[(2-methoxyphenyl)azo]-1-naphthalene sulfonic acid monosodium salt (CI 14710, Acid Red No. 4), 6-Hydroxy-5-[(4-sulfonaphth-1-yl)azo]-2,4-naphthalene disulfonic acid trisodium salt (CI 16255, Ponceau 4R, Acid Red No. 18), 8-Amino-1-hydroxy-2-(phenyl-azo)-3,6-naphthalene disulfonic acid disodium salt (CI 17200, Acid Red No. 33), 5-(Acetylamino)-4-hydroxy-3-[(2-methylphenyl)azo]-2,7-naphthalene disulfonic acid disodium salt (CI 18065, Acid Red No. 35), N-[6-(Diethylamino)-9-(2,4-disulfophenyl)-3H-xanthen-3-yliden]-N-ethylethane ammonium hydroxide, inner salt, sodium salt (CI 45100, Acid Red No. 52), 2′,4′,5′,7′-Tetrabromo-3′,6′-dihydroxyspiro-[isobenzo-furan-1 (3H), 9′-[9H]xanthen]-3-one disodium salt (CI 45380, Acid Red No. 87), 2′,4′,5′,7′-Tetra-bromo-4, 5, 6, 7-tetrachloro-3′,6′-dihydroxyspiro-[isobenzofuran-1 (3H), 9′[9H]xanthen]-3-one disodium salt (CI 45410, Acid Red No. 92), 3′,6′-Dihydroxy-4′,5′-diiodospiro[iso-benzofuran-1(3H), 9′(9H)-xanthen]-3-one disodium salt (CI 45425, Acid Red No. 95), 2-Hydroxy-3-((2-hydroxynaphth-1-yl)azo)-5-nitrobenzene sulfonic acid monosodium salt (CI 15685, Acid Red No. 184), (2-Sulfophenyl)-di-[4-(ethyl-((4-sulfophenyl)methyl)-amino)phenyl]-carbenium disodium salt, betaine (CI 42090, Acid Blue No. 9, FD&C Blue No. 1), 3-Hydroxy-4-((4-methyl-2-sulfo-phenyl)azo)-2-naphthalene carboxylic acid disodium salt (CI 15850, D&C Red No. 6), 6-Hydroxy-5-((2-methoxy-5-methyl-4-sulfophenyl)azo)-2-naphthalene sulfonic acid disodium salt (CI 16035, FD&C Red 40), 1,4-Bis-[(2-sulfo-4-methylphenyl) amino]-9,10-anthraquinone-disodium salt (CI 61570, Acid Green No. 25), 1-Amino-4-(cyclohexylamino)-9,10-anthraquinone-2-sulfonic acid sodium salt (CI 62045, Acid Blue No. 62), 3,3-Bis-(3,5-dibromo-4-hydroxyphenyl)-4,5,6,7-tetrabromo-2,1 (3H)-benzoxathiole-1,1-dioxide, 1-Amino-4-(phenylamino)-9,10-anthraquinon-2-sulfonic acid (CI 62055, Acid Blue No. 25), 1-Hydroxy-4-[(4-methyl-2-sulfophenyl)-amino]-9,10-anthra-quinone sodium salt (CI 60730, D&C Violet No. 2, Acid Violet No. 43), 5-Amino-4-hydroxy-6-[(4-nitrophenyl)azo]-3-(phenylazo)-2,7-naphthalene-disulfonic acid disodium salt (CI 20470, Acid Black No. 1), 3-Hydroxy-4-[(2-hydroxynaphth-1-yl)-azo]-7-nitro-1-naphthalene sulfonic acid chromium complex (3:2) (CI 15711, Acid Black No. 52), 3-[(2,4-Dimethyl-5-sulfophenyl)azo]-4-hydroxy-1-naphthalene sulfonic acid disodium salt (CI 14700, Food Red No. 1, FD&C Red No.4).

Additional known and conventional dyestuffs for hair dyeing, which can be contained in the dye composition according to the invention, are described in, among others, E. Sagrin, “Cosmetics, Science and Technology”, Interscience Publishers Inc., New York (1957), pp. 503 and following, and H. Janistyn, “Handbook of Cosmetics and Perfumes”, Volume 3 (1973), pp. 388 and following and K. Schrader “Foundations and Formulations of Cosmetics”, 2^{nd} Edition (1989), pp. 782-815.

An oxidative dye composition according to the invention is mixed with an oxidizing agent immediately prior to use and the resulting ready-to-apply dye preparation is applied to the hair in an amount sufficient for oxidative dyeing of the hair. In so far as the oxidative dye composition according to the invention contains no oxidation dye precursor compounds and/or oxidation dye precursor compounds that are oxidizable by air oxygen, the dye composition can be applied directly to the hair without first mixing with an oxidizing agent-containing composition. Principally oxidizing agents for developing color comprise hydrogen peroxide or its addition compounds with urea, melamine or sodium borate in the form of a 1 to 12%, preferably 1.5 to 6%, aqueous solution. The mixing ratio of dye composition to oxidizing agent-containing composition depends on the concentration of the oxidizing agent and usually amounts to about 5:1 to 1:2, preferably 1:1. The content of the oxidizing agent in the ready-to-apply dye preparation is preferably about 0.5 to 8 percent by weight, especially 1 to 4 percent by weight.

The composition according to the invention preferably is in the form of an aqueous, alcoholic or aqueous-alcoholic medium preferably with at least 10 percent by weight water. As alcohols suitable for cosmetic purposes lower alcohols with 1 to 4 carbon atoms, such as ethanol and isopropanol, are preferred. The composition according to the invention can have a pH in a range from 2.0 to 9.5 A pH in range between 4 and 8 is especially preferred. Additional co-solvents include organic solvents or a mixture of solvents with a boiling point under 400° C. in an amount of 0.1 to 15 percent by weight, preferably from 1 to 10 percent by weight. Especially suitable co-solvents include branched or unbranched hydrocarbons, such as pentane, hexane, isopentane and cyclic hydrocarbons, such as cyclopentane and cyclohexane. Additional especially preferred water-soluble solvents include glycerol, ethylene glycol and propylene glycol in an amount up to 30 percent by weight.

The compositions according to the invention can also include conventional cosmetic additive ingredients, such as perfume oils, turbidity-imparting agents, such as e.g. ethylene glycol distearate, styrene/PVP copolymers or polystyrene, moisture-containing agents, particulate materials, preservatives; luster-imparting agents, coloring agents, antioxidants, in an amount of preferably 0.01 to 10 percent by weight each, wherein the total amount preferably does not exceed 10 percent by weight.

The composition according to the invention can be in different application forms, e.g. in the form of a hair-care agent, a hair-fixing agent, a hair-cleansing agent, a hair dyeing agent or a tinting agent, a bleaching agent or a permanent shaping agent. The composition can be applied as a lotion, aerosol foam, non-aerosol foam, hair milk, hair gel, liquid gel, spray gel, hair cream, gel-foam, aerosol spray, non-aerosol spray, hair wax or in the form of an emulsion-form hair-care composition (hair rinse, conditioner). Also the composition according to the invention can be in the form of a lotion thickened with a conventional thickener. The composition according to the invention can also be a shampoo containing an anionic, amphoteric or nonionic surfactant or a hair dye composition containing a direct-dyeing or oxidative hair dye, e.g. a dyeing fixing composition.

In various embodiments the composition according to the invention is in the form of a hair gel, in the form of a viscose hair lotion or in the form of a hair spray gel, which is sprayable by a mechanical spraying device, and contains 0.05 to 10, preferably 0.1 to 2, wt. % of at least one thickening polymer and has a viscosity of at least 250 mPa s, as measured with a Bohlin Rheometer CS, measuring body C25 at 25° C. and a shear rate of 50 s¹. The visocity of the gel preferably amounts to 500 to 50,000 mPa s, especially preferably from 1,000 to 15,000 mPa s at 25° C. The thickening polymers can be e.g. copolymers of at least one first type of monomer and at least one second type of monomer. The first type of monomer is an acrylic acid or methacrylic acid. The second type of monomer is selected from esters of acrylic acid and ethoxylated fatty alcohols. The thickening polymers can also be cross-linked polyacrylic acids, cross-linked copolymers of at least one first type of monomer, which is acrylic acid or methacrylic acid, and at least one second type of monomer, which is an ester of acrylic acid with a C₁₀- to C₃₀-alcohol, copolymers of at least one first type of monomer, which is acrylic acid or methacrylic acid, and at least one second type of monomer, which is an ester of itaconic acid and ethoxylated fatty alcohol; copolymers of at least one first type of monomer, which is acrylic acid or methacrylic acid, and at least one second type of monomer, which is an ester of itaconic acid and an ethoxylated C₁₀- to C₃₀-alcohol, and a third type of monomer, which is selected from C₁- to C₄-amino-alkylacrylates; copolymers of two or more monomers, selected from acrylic acid, methacrylic acid, acrylic acid esters and methacrylic acid esters; copolymers of vinyl pyrrolidone and ammonium acryloyl dimethyl taurate; coplymers of ammoniumacryloyldimethyl taurate and monomers selected from esters of methacrylic acid and ethoxylated fatty alcohols; hydroxyethyl cellulose; hydroxypropyl cellulose; hydroxypropyl guar; glyceryl polyacrylate; glyceryl polymethacrylate; copolymers of at least one C₂- to C₄-alkylene and styrene; polyurethanes; hydroxypropyl starch phosphate; polyacrylamides; copolymers of maleic acid anhydride and methyl vinyl ether; St. John's wart meal; guar gum; xanthane; dehydroxyxanthane; carrageenan; karaya gum; hydrolyzed corn starch; copolymers of polyethylene oxide, fatty alcohols and saturated methylenediphenyldiisocyanate (e.g. PEG-150/stearyl alcohol/SMDI copolymer).

Aerosol spray gels are special embodiments of the composition according to the invention. The thickening is achieved by the interaction or reaction of the chitosan derivatives dissolved in the aqueous-ethanolic medium with the propellant gas, e.g. dimethylether. A gel-like mass, which may be easily sprayed from an aerosol container, made of glass or metal, is obtained.

In various embodiments the compositions according to the invention is present in the form of an O/W emulsion, a W/O emulsion or a microemulsion and contains at least one of the above-described oils or waxes emulsified in water and at least one of the above-described surfactants.

In other embodiments the composition according to the invention is in the form of a sprayed product, either in combination with a mechanical pump spray apparatus or in combination with at least one propellant, selected from propane, butane, dimethyl ether and fluorinated hydrocarbons. An aerosol spray contains preferably 15 to 85% by weight, especially preferably from 25 to 75 percent by weight, of a propellant and is filled into a pressurized container. The propellants for example are lower alcohols, e.g. n-butane, i-butane and propane, or their mixtures, and dimethyl ether or fluorohydrocabons, such as F 152a (1,1-difluoroethhane) or F 134 (tetrafluoroethane), and gaseous compounds, such as N₂, N₂O and CO₂, as well as mixture of the above-described propellants.

A non-aerosol hair spray is sprayed with the assistance of a suitable mechanically operated spraying apparatus. A “mechanically operated spray apparatus” is understood to be an apparatus, which permits spraying of a composition without using a propellant. A spray pump or an elastic container provided with a spray valve, in which a cosmetic composition according to the invention is filled under pressure, so that the elastic container is expanded and the composition is dispensed because of its contraction when the spray valve is opened is a suitable mechanically operated spraying apparatus.

In various embodiments the composition according to the invention is in the form of a foamed product (Mousse) in combination with an apparatus for foaming, which contains at least one conventional foam-forming substance known for that purpose, e.g. at least one foam-forming surfactant or at least one foam-forming polymer. The “apparatus for foam formation” is an apparatus, which permits foaming a liquid with or without the use of a propellant. For example, a suitable mechanical foam forming apparatus can be a commercial pump foamer or an aerosol foam head. The product is either in combination with a mechanical pump foam apparatus (pump foamer) or in combination with at least one propellant (aerosol foam) in an amount of preferably 1 to 20, especially fro 2 to 10, percent by weight. Suitable propellants are e.g. selected from propane, butane, dimethyl ether and fluorinated hydrocarbon substances. The composition is foamed immediately prior to application or use and the foam is worked in the hair. Subsequently it can be rinsed out or left on the hair without rinsing.

In some embodiment the composition according to the invention can be in the form of a hair wax, i.e. it has a waxy consistency and contains at least one of the above-mentioned waxes in an amount of preferably from 0.5 to 30 percent by weight and other water-insoluble substances as needed. The waxy consistency is preferably characterized by a needle penetration number of greater than or equal to 10, especially preferably greater than or equal to 20, as measured with a measuring unit of 0.1 mm, test weight 100 g, test duration 5 s, test temperature 25° C., after DIN 51 579. The solidification point of the product is preferably greater than or equal to 30° C. and less than or equal to 70° C., especially preferably in a range from 40° C. to 55° C. Suitable waxes and water-insoluble substances are especially emulsifiers with an HLB value under 7, silicone oils, silicone waxes, waxes (e.g. wax alcohols, wax acids, wax esters, and especially natural waxes, such as beeswax, carnauba wax, etc), fatty alcohols, fatty acids, fatty acid esters or hydrophilic waxes, such as e.g. high molecular weight polyethylene glycols with a molecular weight of 800 to 20,000, preferably from 2,000 to 10,000, g/mol.

When the hair treatment composition according to the invention is present in the form of a hair lotion, it is present as a substantially non-viscous or slightly viscous, fluid solution, dispersion or emulsion with a content of at least 10 percent by weight, preferably 20 to 95 percent by weight, of a cosmetically compatible alcohol. The alcohol especially can be a lower alcohol with 1 to 4 carbon atoms, e.g. ethanol or isopropanol, suitable for cosmetic purposes.

When the hair treatment composition is present in the form of a hair cream, it is present in the form of an emulsion and contains either an additional viscosity-imparting ingredient in an amount of 0.1 to 10 percent by weight or the required viscosity and creamy consistency is built up by micell formation with the help of a suitable surfactant, fatty acid, fatty alcohol, wax, etc in the usual manner.

When the hair treatment composition according to the invention is present as a hair cleansing composition it contains at least one wash-active surfactant, preferably from 0.01 to 25% by weight, especially preferably from 5 to 20 percent by weight, of at least one anionic, amphoteric and/or nonionic surfactant and 50 to 90% by weight water. Suitable surfactants have been already named above. Preferred surfactants for the hair cleansing compositions according to the invention are selected from alkali or alkaline earth salts of C₁₀- to C₁₈-alkyl sulfates, C₁₀- to C₁₈-alkylsulfonates, C₁₀- to C₁₈-alkylbenzene sulfonic acids, C₁₀- to C₁₈-xylene sulfonates, and C₁₀- to C₁₈-alkyl ether sulfates ethoxylated with 1 to 10 ethylene oxide units; ethoxylated sulfosuccinic acid semi-esters of formula R¹(OCH₂CH₂)ₘ—O₂C—CH₂CH(SO₃M)-CO₂M, wherein R¹ is a C₁₀- to C₁₈-alkyl group, M is an alkali metal or alkaline earth methal cation and m is a whole number from 1 to 10; alkyl ether carboxylates of formula: R²(OCH₂CH₂)ₙ—OCH₂—COOM, wherein R² is a C₁₀- to C₁₈-alkyl group, M is an alkali metal or alkaline earth methal cation and n is a whole number from 1 to 10; ethoxylated fatty alcohols with 12 to 18 carbon atoms; polyglyceryl ethers of staturated or unsaturated fally alcohols and alkyl phenols with 8 to 30 carbon atoms in the alkyl group and 1 to 10 glyceryl units in the molecule; fatty acid alkanol amies; ethoxylate sorbitan fatty acid esters, C₈- to C₁₈-alkylpolyglucosides and C₈- to C₁₈-alkylbetaines. Preferred amphoteric surfactants are of the general formula (V):
R²—Y⁽⁺⁾(R³)ₓ—CH₂—R⁴Z⁽⁻⁾ (V),
wherein R² denotes a linear or branched alkyl-, alkenyl- or hydroxyalkyl group with 8 to 18 carbon atoms and 0 to 10 ethylene oxide units and 0 to 1 glyceryl units; Y denotes an N-, P-, or S atom; R³ is an alkyl or monohydroxyalkyl group with 1 to 3 carbon atoms; x is 1, in case Y is an S atom and x is 2 when Y is an N or a P atom; R⁴ denotes an alkylene or hydroxyalkyl group with 1 to 4 carbon atoms and Z denotes a carboxylate, sulfate, phosphonate or phosphate group.

The subject matter of the invention is also a method of treating hair, comprising
preparing a hair treatment composition according to the invention,
applying the hair treatment composition to hair, and
leaving the hair treatment composition on the hair or rinsing out after an acting time.

If the cosmetic composition according to the invention is used for hair care or hair fixing, the following steps are employed: After washing the hair from 5 to 30 g of the composition, according to the hair abundance, is distributed on the hand towel dried hair

The following examples illustrate the above-described invention in more detail, but the details in these examples should not be considered as limiting the claims appended hereinbelow.

### EXAMPLES

The N-hydroxypropyl-O-benzyl chitosan (HPBC) used in these examples is made by methods similar to those described in EP 0 300 234 by reaction of a chitosan with a molecular mass between 300,000 and 700,000 g/mol and an acetylation degree between 0.1 and 0.3 with 2 to 7 equivalents of propylene oxide and 0.25 to 1.5 equivalents of benzyl chloride.

### EXAMPLE 1

### Rinse-Out Hair Care Foam Compositions of the Invention and Prior Art

| Ingredient | A | B | C | D | V |
|---|---|---|---|---|---|
| HPBC | 0.50 | 1.50 | 2.50 | 4.00 | — |
| PEG-35 castor oil | 0.3 | 0.3 | 0.3 | 0.3 | 0.3 |
| PEG-4 lauryl ether | 0.3 | 0.3 | 0.3 | 0.3 | 0.3 |
| Cetrimonium chloride | 0.1 | 0.1 | 0.1 | 0.1 | 0.1 |
| Isopropanol | 0.07 | 0.07 | 0.07 | 0.07 | 0.07 |
| Ethanol | 10 | 10 | 10 | 10 | 10 |
| Polyquaternium-16* | 1.4 | 1.4 | 1.4 | 1.4 | 1.4 |
| Perfume oil | 0.3 | 0.3 | 0.3 | 0.3 | 0.3 |
| Water | To 100 | To 100 | To 100 | To 100 | To 100 |

*vinylimidazoliminium methochloride/vinyl pyrrolidone copolymer

96 g of the hair care composition are filled into an aerosol container with 4 g of a mixture of 50% by weight propane and 50% by weight n-butane. The care foam according to the invention is dispensed from the container in foam form by spraying.

In comparison with the comparative composition V of the prior art the compositions A to D according to the invention are characterized by
increased elasticity of the hairdo
increased volume of the hairdo
improved feel and combability of moist and dry hair.

### EXAMPLE 2

### Volume Shampoo Compositions of the Invention and Prior Art

| Ingredient | A | B | C | D | V |
|---|---|---|---|---|---|
| HPBC | 0.50 | 1.50 | 2.50 | 4.00 | — |
| Sodium lauryl ether | 2.7 | 2.7 | 2.7 | 2.7 | 2.7 |
| sulfate | | | | | |
| Benzoic acid | 0.03 | 0.03 | 0.03 | 0.03 | 0.03 |
| Cocconut fatty acid | 1.5 | 1.5 | 1.5 | 1.5 | 1.5 |
| amidopropyl betaine | | | | | |
| Ethylene glycol | 2 | 2 | 2 | 2 | 2 |
| distearate | | | | | |
| Perfume oil | 0.4 | 0.4 | 0.4 | 0.4 | 0.4 |
| Sodium chloride | 3.3 | 3.3 | 3.3 | 3.3 | 3.3 |
| Water | To 100 | To 100 | To 100 | To 100 | To 100 |

In comparison with the comparative composition V of the prior art the compositions A to D according to the invention are characterized by
increased elasticity of the hairdo
increased volume of the hairdo
improved feel and combability of moist and dry hair.

### EXAMPLE 3

### Hairstyling Foam Compositions of the Invention and Prior Art

| Ingredient | A | B | C | D | V |
|---|---|---|---|---|---|
| HPBC | 0.50 | 1.50 | 2.50 | 4.00 | 0.00 |
| Polyquaternium-11 | 6.5 | 4.5 | 3.0 | 2.5 | 8 |
| Cetrimonium chloride | 0.1 | 0.1 | 0.75 | 0.5 | 0.2 |
| Ethanol | 0.5 | 0.5 | 0.5 | 0.5 | 0.5 |
| Water | To 100 | To 100 | To 100 | To 100 | To 100 |

In comparison with the comparative composition V of the prior art the compositions A to D according to the invention are characterized by
reduced load
improved luster
improved fixing.

### EXAMPLE 4

### Hair Spray Gels of the Invention and Prior Art

| Ingredient | A | B | C | D | V |
|---|---|---|---|---|---|
| HPBC | 0.1 | 0.5 | 1.5 | 2.5 | — |
| Acrylates copolymer | 2.00 | 2.10 | — | — | 2.50 |
| Carbomer | — | — | 0.08 | 0.10 | — |
| Aminomethylpropanol | 0.17 | 0.15 | 0.08 | 0.11 | 0.18 |
| Glycerol | 0.10 | 0.10 | 0.10 | 0.10 | 0.10 |
| PEG-40 hydrogenated | 0.02 | 0.02 | 0.02 | 0.02 | 0.02 |
| castor oil | | | | | |
| Perfume | 0.02 | 0.02 | 0.02 | 0.02 | 0.02 |
| Ethanol | 25 | 25 | 25 | 25 | 25 |
| Polyvinyl pyrrolidone/ | 2.50 | 5.00 | 2.50 | 3.20 | 0.00 |
| Vinyl acetate copolymer | | | | | |
| Polyvinyl pyrrolidone | 0.00 | 0.00 | 0.00 | 0.00 | 2.60 |
| Water | To 100 | To 100 | To 100 | To 100 | To 100 |

### EXAMPLE 5

### Hair Balm Compositions

| Ingredient | A | B | C | D |
|---|---|---|---|---|
| HPBC | 0.5 | 1.50 | 2.50 | 4.00 |
| Glycerl stearate/polyethylene | 6 | 6 | 6 | 6 |
| glycol-(20)-cetearyl ether | | | | |
| Diquaternary polydimethyl- | 4 | 4 | 4 | 4 |
| siloxane (ABIL ® Quat 3272) | | | | |
| Cetyl alcohol | 2 | 2 | 2 | 2 |
| Citric acid | 1.36 | 1.36 | 1.36 | 1.36 |
| Preservative, Perfume | q.s | q.s | q.s | q.s |
| Water | To 100 | To 100 | To 100 | To 100 |

### EXAMPLE 6

### Hair Rinses

| Ingredient | A | B | C | D |
|---|---|---|---|---|
| HPBC | 0.5 | 1.50 | 2.50 | 4.00 |
| Cetyl stearyl alcohol | 4 | 4 | 4 | 4 |
| Pyrrolidone carboxylic acid | 1.36 | 1.36 | 1.36 | 1.36 |
| Cetyltrimethyl ammonium | 0.75 | 0.75 | 0.75 | 0.75 |
| chloride | | | | |
| Perfume | 0.5 | 0.5 | 0.5 | 0.5 |
| Plant extract EXTRAPON ® 5 | 0.2 | 0.2 | 0.2 | 0.2 |
| Special | | | | |
| Water | To 100 | To 100 | To 100 | To 100 |

### EXAMPLE 7

### Sprayed Hair Care Compositions

| Ingredient | A | B | C | D |
|---|---|---|---|---|
| HPBC | 0.5 | 1.50 | 2.50 | 4.00 |
| Poly(dimethyldiallyl ammonium | 2 | 2 | 2 | 2 |
| chloride) | | | | |
| Polyethylene glycol-(40)- | 1.25 | 1.25 | 1.25 | 1.25 |
| Sorbitan monopalmitate | | | | |
| Pyrrolidone carboxylic acid | 1 | 1 | 1 | 1 |
| Perfume | 0.1 | 0.1 | 0.1 | 0.1 |
| Cetyltrimethyl ammonium chloride | 0.03 | 0.03 | 0.03 | 0.03 |
| Ethanol | 15 | 15 | 15 | 15 |
| Water | To 100 | To 100 | To 100 | To 100 |

### EXAMPLE 8

### Foamed Hair Care Compositions

| Ingredient | A | B | C | D |
|---|---|---|---|---|
| HPBC | 0.5 | 1.50 | 2.50 | 4.00 |
| Dow Corning 929 | 2 | 2 | 2 | 2 |
| (Amodimethicone, 35%) | | | | |
| Citric acid | 1.3 | 1.3 | 1.3 | 1.3 |
| Hydroxypropyl cellulose | 0.5 | 0.5 | 0.5 | 0.5 |
| (M = 1,150,000 g/mol) | | | | |
| Dow Corning 2501 | 0.3 | 0.3 | 0.3 | 0.3 |
| Cosmetic Wax* | | | | |
| Perfume | 0.2 | 0.2 | 0.2 | 0.2 |
| Cetyltrimethyl | 0.25 | 0.25 | 0.25 | 0.25 |
| ammonium chloride | | | | |
| D-panthenol | 0.15 | 0.15 | 0.15 | 0.15 |
| Elastin hydrolyzate | 0.1 | 0.1 | 0.1 | 0.1 |
| Propane/butane, 5.0 bar | 5 | 5 | 5 | 5 |
| Ethanol | 10 | 10 | 10. | 10 |
| Water | To 100 | To 100 | To 100 | To 100 |

*Bis-PEG-18 methyl ether dimethylsilane

### EXAMPLE 9

### Hair Fixing Compositions

| Ingredient | A | B | C | D |
|---|---|---|---|---|
| HPBC | 0.1 | 0.5 | 1.50 | 2.5 |
| Vinyl pyrrolidone/vinyl | 3 | 3 | 3 | 3 |
| acetate copolymer | | | | |
| 1,2 Propylene glycol | 0.2 | 0.2 | 0.2 | 0.2 |
| Perfume | 0.15 | 0.15 | 0.15 | 0.15 |
| Cetyltrimethyl ammonium | q.s. | q.s. | q.s. | q.s. |
| chloride | | | | |
| Water | 20 | 20 | 20 | 20 |
| Ethanol | To 100 | To 100 | To 100 | To 100 |

### EXAMPLE 10

### Foam Fixing Agents

| Ingredient | A | B | C | D |
|---|---|---|---|---|
| HPBC | 0.1 | 0.5 | 1.5 | 2.5 |
| Polyvinyl pyrrolidone | 3.15 | 3.15 | 3.15 | 3.15 |
| Citric acid | 1.6 | 1.6 | 1.6 | 1.6 |
| Hydrogenated castor oil, | 0.6 | 0.6 | 0.6 | 0.6 |
| ethoxylated with 40 mol | | | | |
| ethylene oxide | | | | |
| Decylpolyglucoside | 0.22 | 0.22 | 0.22 | 0.22 |
| Vinylpyrrolidone/methacryl- | 0.2 | 0.2 | 0.2 | 0.2 |
| amidopropyltrimthyl ammonium | | | | |
| chloiride copolymer | | | | |
| Perfume | 0.2 | 0.2 | 0.2 | 0.2 |
| Propane/butane, 5.0 bar | 6 | 6 | 6 | 6 |
| Water | To 100 | To 100 | To 100 | To 100 |

### EXAMPLE 11

### Hair Sprays

| Ingredient | A | B | C | D |
|---|---|---|---|---|
| HPBC | 0.1 | 0.5 | 1.5 | 2.5 |
| Acrylates/acrylamide copolymer | 3.5 | 3.5 | 3.5 | 3.5 |
| Water | 3.5 | 5 | 6 | 10 |
| Perfume | 0.15 | 0.15 | 0.15 | 0.15 |
| Dimethyl ether | 45 | 45 | 45 | 45 |
| Ethanol | To 100 | To 100 | To 100 | To 100 |

### EXAMPLE 12

### 80% VOC Hair Sprays

| Ingredient | A | B | C | D |
|---|---|---|---|---|
| HPBC | 0.1 | 0.5 | 1.5 | 2.5 |
| t-butylacrylate/ethylacrylate/ | 4 | 4 | 4 | 4 |
| methacrylic acid terpolymer | | | | |
| 2-amino-2-methyl-1-propanol | 0.72 | 0.72 | 0.72 | 0.72 |
| Cyclo-tetra(dimethylsiloxane) | 0.2 | 0.2 | 0.2 | 0.2 |
| Perfume | 0.05 | 0.05 | 0.05 | 0.05 |
| Water | 15 | 15 | 15 | 15 |
| Dimethyl ether | 40 | 40 | 40 | 40 |
| Ethanol | To 100 | To 100 | To 100 | To 100 |

### EXAMPLE 13

### Pump Hair Sprays

| Ingredient | A | B | C | D |
|---|---|---|---|---|
| HPBC | 0.1 | 0.5 | 1.5 | 3.5 |
| Vinyl pyrrolidone/vinyl | 4 | 4 | 4 | 4 |
| acetate copolymer | | | | |
| Ethanol | 28.5 | 28.5 | 28.5 | 28.5 |
| Perfume | 0.25 | 0.25 | 0.25 | 0.25 |
| Cetyltrimethyl ammonium | 0.2 | 0.2 | 0.2 | 0.2 |
| chloride | | | | |
| Water | To 100 | To 100 | To 100 | To 100 |

### EXAMPLE 14

### Hair Dyeing-Fixing Compositions

| Ingredient | A | B | C |
|---|---|---|---|
| HPBC | 0.50 | 1.50 | 2.50 |
| Vinyl pyrrolidone/vinyl acetate | 3.5 | 3.5 | 3.5 |
| copolymer | | | |
| Perfume | 0.2 | 0.2 | 0.2 |
| 1-amino-4-(2′,3′-dehydropropyl)amino- | 0.07 | 0.07 | 0.07 |
| 5-chloro-2-nitrobenzene | | | |
| Basic Brown 17 (C.I. 12 251) | 0.05 | 0.05 | 0.05 |
| Basic Blue 7 (C.I. 42 595) | 0.01 | 0.01 | 0.01 |
| Basic Violet 14 (C.I. 42510) | 0.0023 | 0.0023 | 0.0023 |
| Ethanol | 50 | 50 | 50 |
| Water | To 100 | To 100 | To 100 |

### EXAMPLE 15

### Hair Care Rinses

| Ingredient | A | B | C | D |
|---|---|---|---|---|
| HPBC | 0.5 | 1.50 | 2.50 | 4.00 |
| Cetylstearyl alcohol | 2.5 | 2.5 | 2.5 | 2.5 |
| Lauryl alcohol diglycol ether | 0.8 | 0.8 | 0.8 | 0.8 |
| VASELINE ® | 1.1 | 1.1 | 1.1 | 1.1 |
| Cetylstearyl sulfate, sodium salt | 0.4 | 0.4 | 0.4 | 0.4 |
| Betaine monohydrate | 5 | 5 | 5 | 5 |
| Perfume oil | 0.2 | 0.2 | 0.2 | 0.2 |
| Water | To 100 | To 100 | To 100 | To 100 |

### EXAMPLE 16

### Hair Care Compositions, not to be Rinsed Out

| Ingredient | A | B | C |
|---|---|---|---|
| HPBC | 0.50 | 1.50 | 2.50 |
| Distearyldimethylammonium chloride | 0.6 | 0.6 | 0.6 |
| Glycerol monodistearate | 0.2 | 0.2 | 0.2 |
| Polyoxyethylene-(45)-polyoxypropylene- | 0.15 | 0.15 | 0.15 |
| (33)-monobutyl ether | | | |
| Stearyl alcohol | 0.1 | 0.1 | 0.1 |
| Stearyl betaine | 0.09 | 0.09 | 0.09 |
| p-hydroxybenzoic acid methyl ester | 0.1 | 0.1 | 0.1 |
| Water | To 100 | To 100 | To 100 |

### EXAMPLE 17

### Hairstyling Gels

| Ingredient | A | B | C | D |
|---|---|---|---|---|
| HPBC | 0.5 | 1.50 | 2.50 | 4.00 |
| Polyvinyl pyrrolidone | 3 | 3 | 3 | 3 |
| Polyacrylic acid | 0.5 | 0.5 | 0.5 | 0.5 |
| (carbomer) | | | | |
| Aminomethylpropanol | 0.39 | 0.39 | 0.39 | 0.39 |
| Ethanol | 12 | 12 | 12 | 12 |
| Water | To 100 | To 100 | To 100 | To 100 |

### EXAMPLE 18

### Hair Sprays

| Ingredient | A | B | C |
|---|---|---|---|
| HPBC | 0.50 | 1.50 | 2.50 |
| Vinyl pyrrolidone/vinyl acetate | 2.5 | 2.5 | 2.5 |
| copolymer | | | |
| Acrylates/acrylamide copolymer* | 2.5 | 2.5 | 2.5 |
| Perfume oil | 0.2 | 0.2 | 0.2 |
| n-butane | 60 | 60 | 60 |
| Ethanol | To 100 | To 100 | To 100 |

*ULTRAHOLD ® 8, neutralized with aminomethylpropanol

### EXAMPLE 19

### Sprayed Hair Care Compositions, not to be Rinsed Out

| Ingredient | A | B | C | D |
|---|---|---|---|---|
| HPBC | 0.50 | 1.50 | 2.50 | 4.00 |
| Polyvinyl pyrrolidone | 0.8 | 0.8 | 0.8 | 0.8 |
| Cetyltrimethyl ammonium chloride | 0.4 | 0.4 | 0.4 | 0.4 |
| Cocamidopropyl betaine | 0.33 | 0.33 | 0.33 | 0.33 |
| Citric acid | 0.2 | 0.2 | 0.2 | 0.2 |
| Isopropanol | 0.28 | 0.28 | 0.28 | 0.28 |
| Keratin hydroyzate | 0.1 | 0.1 | 0.1 | 0.1 |
| Water | To 100 | To 100 | To 100 | To 100 |

### EXAMPLE 20

### Sprayed Hair Care Compositions, not to be Rinsed Out

| Ingredient | A | B | C |
|---|---|---|---|
| Spherical microparticles | 0.50 | 1.50 | 2.50 |
| Polyvinyl pyrrolidone | 0.9 | 0.9 | 0.9 |
| Vinylimidazolinium methochloride/ | 0.32 | 0.32 | 0.32 |
| vinylpyrrolidone copolymer | | | |
| Cetyltrimethyl ammonium chloride | 0.4 | 0.4 | 0.4 |
| Coconut fatty acid amidopryopyl betaine | 0.21 | 0.21 | 0.21 |
| PEG-7 hydrogenated castor oil | 0.05 | 0.05 | 0.05 |
| PEG-40 hydrogenated castor oil | 0.18 | 0.18 | 0.18 |
| Keratin hydroyzate | 0.2 | 0.2 | 0.2 |
| Ethanol | 12 | 12 | 12 |
| Glyoxylic acid | 0.2 | 0.2 | 0.2 |
| Water | To 100 | To 100 | To 100 |

### EXAMPLE 21

### Permanent Shaping Compositions

| Ingredient | A | B | C | D |
|---|---|---|---|---|
| HPBC | 0.5 | 1.50 | 2.50 | 4.00 |
| Thioglycolic acid | 8.82 | 8.82 | 8.82 | 8.82 |
| PEG-35 castor oil | 0.90 | 0.90 | 0.90 | 0.90 |
| Polyethylene glycol-(23)- | 0.90 | 0.90 | 0.90 | 0.90 |
| nonylphenyl ether | | | | |
| Poly(diallyldimethyl- | 0.90 | 0.90 | 0.90 | 0.90 |
| Ammonium chloride) | | | | |
| Styrene/polyvinyl | 0.28 | 0.28 | 0.28 | 0.28 |
| pyrrolidone copolymer | | | | |
| Ammonia | 0.15 | 0.15 | 0.15 | 0.15 |
| Ammonium hydrogen | 3.5 | 3.5 | 3.5 | 3.5 |
| carbonate | | | | |
| Perfume oil | 0.5 | 0.5 | 0.5 | 0.5 |
| Water | To 100 | To 100 | To 100 | To 100 |

### EXAMPLE 22

### Fixing Agents for Permanent Shaping

| Ingredient | A | B | C | D |
|---|---|---|---|---|
| HPBC | 0.5 | 1.50 | 2.50 | 4.00 |
| Cetylstearyl alcohol | 2.61 | 2.61 | 2.61 | 2.61 |
| Sodium lauryl sulfate | 0.29 | 0.29 | 0.29 | 0.29 |
| Lanolin alcohol | 0.4 | 0.4 | 0.4 | 0.4 |
| Hydrogen peroxide | 1.9 | 1.9 | 1.9 | 1.9 |
| Phosphoric acid | 0.085 | 0.085 | 0.085 | 0.085 |
| Salicylic acid | 0.1 | 0.1 | 0.1 | 0.1 |
| Perfume oil | 0.3 | 0.3 | 0.3 | 0.3 |
| Water | To 100 | To 100 | To 100 | To 100 |

### EXAMPLE 23

### Foam Fixing Agents

| Ingredient | A | B | C | D |
|---|---|---|---|---|
| Spherical microparticles | 0.5 | 1.50 | 2.50 | 4.00 |
| Hydrogen peroxide (35%) | 14 | 14 | 14 | 14 |
| o-phosphoric acid (85%) | 3.41 | 3.41 | 3.41 | 3.41 |
| DL-2-pyrrolidone-5-carboxylic | 1.25 | 1.25 | 1.25 | 1.25 |
| acid | | | | |
| Laurylaminodimetylacetobetaine | 0.66 | 0.66 | 0.66 | 0.66 |
| Polypropylene-(1)-poly- | 0.5 | 0.5 | 0.5 | 0.5 |
| ethylene-(9) lauryl glycol | | | | |
| Perfume | 0.2 | 0.2 | 0.2 | 0.2 |
| p-acetaminophenol | 0.05 | 0.05 | 0.05 | 0.05 |
| Water | To 100 | To 100 | To 100 | To 100 |

### EXAMPLE 24

### Hair Tinting Foams

| Ingredient | A | B | C | D |
|---|---|---|---|---|
| HPBC | 0.5 | 1.50 | 2.50 | 4.00 |
| Cetylstearyl alcohol | 4.5 | 4.5 | 4.5 | 4.5 |
| Cetyltrimonium ammonium | 1.75 | 1.75 | 1.75 | 1.75 |
| chloride | | | | |
| Isopropanol | 4.2 | 4.2 | 4.2 | 4.2 |
| Basic Violet 14 (C.I. 42 510) | 0.0025 | 0.0025 | 0.0025 | 0.0025 |
| Water | To 100 | To 100 | To 100 | To 100 |

94.00 g of the above-described hair tinting foam are filled in an aerosol container with 6 g of a mixture of 50 wt. % propane, 40 wt % n-butane and 10 wt. % dimethyl ether. The hair tinting agent according to the invention is dispensed from the container in foam form by spraying.

### EXAMPLE 25

### Oxidative Hair Dye Compositions (Dark Brown)

| Ingredient | A | B | C | D |
|---|---|---|---|---|
| HPBC | 0.5 | 1.50 | 2.50 | 4.00 |
| Cetylstearyl alcohol | 17.1 | 17.1 | 17.1 | 17.1 |
| Sodium lauryl sulfate | 1.9 | 1.9 | 1.9 | 1.9 |
| Lanolin alcohol | 2.1 | 2.1 | 2.1 | 2.1 |
| Glyceryl stearate | 6.1 | 6.1 | 6.1 | 6.1 |
| Sodium cocoyl isoethionate | 0.4 | 0.4 | 0.4 | 0.4 |
| Sodium lauryl ether sulfate | 1.4 | 1.4 | 1.4 | 1.4 |
| p-aminophenol hydrochloride | 0.06 | 0.06 | 0.06 | 0.06 |
| p-toluylenediaminosulfate | 0.65 | 0.65 | 0.65 | 0.65 |
| Resorcinol | 0.26 | 0.26 | 0.26 | 0.26 |
| Sodium sulfite | 0.6 | 0.6 | 0.6 | 0.6 |
| Ethylenediaminetetraacetic acid | 0.3 | 0.3 | 0.3 | 0.3 |
| Isopropanol | 6 | 6 | 6 | 6 |
| Perfume oil | 0.3 | 0.3 | 0.3 | 0.3 |
| Ammonia | 1.4 | 1.4 | 1.4 | 1.4 |
| Water | To 100 | To 100 | To 100 | To 100 |

Prior to application 50 g of the above-described composition is mixed with 50 ml of hydrogen peroxide (6% solution).

### EXAMPLE 26

### Aerosol Fixing Gels

| Ingredient | A | B | C | D |
|---|---|---|---|---|
| HPBC | 2.00 | 3.00 | 3.50 | 4.00 |
| Polyvinyl pyrrolidone | 5.00 | — | | 3.00 |
| Vinyl pyrrolidone/vinyl | — | 3.50 | — | — |
| acetate | | | | |
| Acrylates/acrylamide | — | — | 3.00 | — |
| Copolymer* | | | | |
| PEG-40 hydrogenated | 0.15 | 0.20 | 0.15 | 0.25 |
| castor oil | | | | |
| Ethanol | 40.00 | 40.00 | 45.00 | 40.00 |
| Ceteareth-12 | 0.15 | 0.20 | 0.20 | 0.25 |
| Perfume | 0.2 | 0.3 | 0.25 | 0.30 |
| Dimethyl ether | 35 | 35 | 30 | 30 |
| Water | To 100 | To 100 | To 100 | To 100 |

*ULTRAHOLD ® 8, neutralized with aminomethylpropanol

The application occurs from a glass aerosol or aluminum aerosol container with a suitable spray valve.

The disclosure in German Patent Application 10 2004 017 431.8 of Apr. 8, 2004 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in stable foamed compositions, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A hair treatment composition containing at least one N-hydroxyalkyl-O-benzyl chitosan and at least one hair treatment effective ingredient.

2. The hair treatment composition as defined in claim 1, wherein said at least one N-hydroxyalkyl-O-benzyl chitosan has at least one hydroxylalkyl group and from 2 to 20 carbon atoms.

3. The hair treatment composition as defined in claim 2, wherein said at least one hydroxyalkyl group is a hydroxylethyl group, a hydroxylpropyl group or a hydroxybutyl group.

4. The hair treatment composition as defined in 1, wherein said at least one N-hydroxyalkyl-O-benzyl chitosan is obtained by N-hydroxyalkylation of chitosan with an alkylene oxide and simultaneously or subsequently O-benzylation with a reactive benzyl compound.

5. The hair treatment composition as defined in claim 4, wherein said alkylene oxide is ethylene oxide, propylene oxide and/or butylene oxide and/or said benzyl compound is a benzyl halide.

6. The hair treatment composition as defined in 1, wherein said at least one N-hydroxyalkyl-O-benzyl chitosan has at least one unit of formula (I): 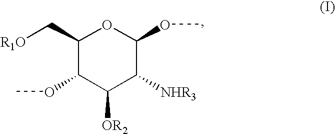
wherein R¹ and R², independently of each other, denote a -(AO)ₙ—H group, A denotes a C₂- to C₂₀-alkylene group; n is a number greater than or equal to zero and R³ denotes H, —CH₂Ph or —CO—CH₃, with the proviso that n is greater than 0 and R³ equals —CH₂Ph in at least one of said at least one unit and/or that n is greater than zero and R³ equals H or —CO—CH₃ in at least one first unit and n equals zero and R³ equals —CH₂Ph for at least one second unit.

7. The hair treatment composition as defined in claim 1, wherein said at least one hair treatment effective ingredient is a hair-care effective ingredient, a hair protecting effective ingredient, a hair repair effective ingredient, a hair cleansing effective ingredient, a hair dyeing ingredient, a hair-fixing effective ingredient and a hair shaping effective ingredient.

8. The hair treatment composition as defined in claim 1, wherein said at least one hair treatment effective ingredient is selected from the group consisting of hair care surfactants, hair cleaning surfactants, hair care oils, hair care waxes, hair-care polymers, hair care silicone compounds, hair-fixing polymers, light protective agents, oxidation hair dye precursors, direct-dyeing dye compounds, hair dye pigments, oxidizing agents and keratin-reducing substances.

9. The hair treatment composition as defined in claim 1, containing from 0.01 to 20 percent by weight of said at least one N-hydroxyalkyl-O-benzyl chitosan and from 0.01 to 20 percent by weight of said at least one hair treatment effective ingredient.

10. The hair treatment composition as defined in claim 1, further comprising at least one polymer with an anionic or anionizable group as hair-fixing polymer or hair-care polymer, and wherein said at least one polymer with at least one anionic or anionizable group is selected from terpolymers of acrylic acid, ethyl acrylate and N-tert.-butylacrylamide; cross-linked or uncross-linked vinyl acetate/crotonic acid copolymers, terpolymers of tert.-butyl acrylate, ethyl acrylate and methacrylic acid; sodium polystyrene sulfonate; copolymers of vinyl acetate, crotonic acid and vinyl propionate; copolymers of vinyl acetate, crotonic acid and vinyl neodeconoate; aminomethylpropanol acrylate copolymers; copolymers of vinyl pyrrolidone and at least one of acrylic acid, methacrylic acid, acrylic acid esters and methacrylic acid esters; copolymers of methyl vinyl ether and maleic acid monoalkyl esters; aminomethylpropanol salts of copolymers of allyl methacrylate and at least one of acrylic acid, methacrylic acid, acrylic acid esters and methacrylic acid esters; cross-linked copolymers of ethyl acrylate and methacrylic acid; copolymers of vinyl acetate, mono-n-butyl maleate and isobornyl acrylate; copolymers of two or more of acrylic acid, methacrylic acid, acrylic acid esters and methacrylic acid esters; copolymers of octylacrylamide and at least one of acrylic acid, methacrylic acid, acrylic acid esters and methacrylic acid esters and polyesters of diglycol, cyclohexane dimethanol, isophthalic acid and sulfoisophthalic acid.

11. The hair treatment composition as defined in claim 1, further comprising at least one polymer with a cationic or cationizable group as hair-fixing polymer or hair-care polymer, and wherein said at least one polymer with at least one cationic or cationizable group is selected from the group consisting of cationic cellulose derivatives of hydroxyethyl cellulose and diallyldimethylammonium chloride; cationic cellulose derivatives of hydroxyethyl cellulose and trimethyl ammonium substituted epoxides; poly(dimethyldiallylammonium chloride); copolymers of acrylamides and dimethyldiallylammonium chloride; quaternary ammonium polymers formed by reaction of diethyl sulfate and a copolymer of vinyl pyrrolidone and dimethylaminoethylmethacrylate; quaternary ammonium polymers of methylvinylimidazolium chloride and vinyl pyrrolidone; polyquaternium-35; polymers of trimethylammoniumethylmethacrylate chloride; polyquaternium-57; substituted polydiemthylsiloxanes terminated with quaternary ammonium groups; copolymers of vinyl pyrrolidone, dimethylaminopropylmethacrylamide and methacryloylaminopropyllauryldimethylammonium chloride; chitosan; chitosan salts; hydroxyalkyl chitosans, hydroxyalkyl chitosan salts, alkylhydroxyalkyl chitosans, alkylhydroxyalkyl chitosan salts, N-hydroxyalkyl chitosanalkyl ether; copolymers of vinyl caprolactam, vinyl pyrrolidone and dimethylaminoethylmethacrylate; copolymers of vinyl pyrrolidone and dimethylaminoethylmethacrylate; copolymers of vinyl pyrrolidone, vinyl caprolactam and dimethylaminopropylacrylamide and polyesters built up from at least one monomer compound; wherein said at least one monomer compound is a polyhydroxy acid substituted with at least one quaternary ammonium group.

12. The hair treatment composition as defined in claim 1, further comprising at least one zwitterionic or amphoteric polymer as hair-fixing polymer or hair-care polymer, and wherein said at least one zwitterionic and/or amphoteric polymer is selected from the group consisting of copolymers of octylacrylamide, acrylic acid, butylaminoethylmethacrylate, methylmethacrylate and hydroxypropylmethacrylate; copolymers of lauryl acrylate, stearyl acrylate, ethylaminoxide methacrylate and at least one of acrylic acid, methacrylic acid, acrylic acid esters and methacrylic acid esters; copolymers of methacryloylethylbetaine and at least one of methacrylic acid and methacrylic acid esters; copolymers of acrylic acid, methacrylates and methacrylamidopropyltrimethylammonium chloride; polymers made from quaternary crotonic betaines; and polymers made from quaternary crotonic betaine esters.

13. The hair treatment composition as defined in claim 1, further comprising at least one nonionic polymer as hair-fixing polymer or hair-care polymer and wherein said at least one nonionic polymer is selected from the group consisting of polyvinyl pyrrolidone, polyvinyl caprolactam, vinyl pyrrolidone/vinyl acetate copolymer, polyvinyl alcohol, isobutylene/ethylmaleimide/hydroxyethyl maleimide copolymer; and copolymers of vinyl pyrrolidone, vinyl acetate and vinyl propionate.

14. The hair treatment composition as defined in claim 1, further comprising at least one silicone compound as a hair-care silicone agent, and where said at least one silicone compound is selected from the group consisting of cyclic dimethylsiloxanes, linear polydimethylsiloxanes, block polymers from polydimethylsiloxanes and polyethylene oxides and/or polypropylene oxides, polydimethylsiloxanes with side chain and terminal polyethylene oxides or polypropylene oxide groups, polydimethylsiloxanes with terminal hydroxyl groups, phenyl substituted polydimethylsiloxanes, silicone emulsions, silicone elastomers, silicone waxes, silicone gum, amino-substituted silicones and silicones substituted with one or more quaternary ammonium group.

15. The hair treatment composition as defined in claim 1, further comprising at least one light protective agent, and where said at least one light protective agent is selected from the group consisting of 4-methoxycinamic acid-2-ethylhexyl ester, methylmethoxy cinnamate, 2-hydroxy-4-methoxybenzophenon-5-sulfonic acid and polyethoxylated p-aminobenzoate.

16. The hair treatment composition as defined in claim 1, further comprising at least one additive ingredient comprising oil acting as hair-care oil or a wax acting as hair-care or hair-fixing wax, and wherein said at least one additive ingredient is selected from the group consisting of paraffin waxes, polyolefin waxes, wool wax, wool wax alcohol, candelilla wax, olive wax, carnauba wax, Japan wax, apple wax, hardened fats, fatty acid esters, fatty acid glycerides, fatty acid triglycerides, polyethylene glycol waxes, silicone waxes, silicone oils, mineral oils, isoparaffin oils, paraffin oils, squalane, sunflower seed oil, coconut oil, castor oil, lanolin oil, jojoba oil, corn oil and soybean oil.

17. The hair treatment composition as defined in claim 1, further comprising at least one hair-protecting or -repairing ingredient, and wherein said at least one hair-protecting or -repairing ingredient is selected from the group consisting of betaine, panthenol, panthenyl ethyl ether, sorbitol, protein hydroylyzates, plant extracts, A-B block copolymers of alkyl acrylates and alkylmethacrylates, A-B block copolymers of alkylmethacrylates and acryl nitrile, A-B-A block copolymers from lactide and ethylene oxide, A-B-A block copolymers of caprolactone and ethylene oxide, A-B-C block copolymers of alkylene or alkadiene compounds, styrene and alkylmethacrylates, A-B-C block copolymers of acrylic acid, styrene and alkylmethacrylates, star-form block copolymers, hyper-branched polymers, dendrimers, intrinsically electrically conductive 3,4-polyethylenedioxythiophenes and intrinsically electrically conductive polyanilines.

18. The hair treatment composition as defined in claim 1, further comprising at least one hair-care surfactant, and wherein said at least one hair-care surfactant is selected from the group consisting of addition products of 2 to 30 mol ethylene oxide and/or 1 to 5 mol propylene oxide to C₈- to C₂₂-fatty alcohols, addition products of 2 to 30 mol ethylene oxide and/or 1 to 5 mol propylene oxide to C₁₂- to C₂₂-fatty acids, addition products of 2 to 30 mol ethylene oxide and/or 1 to 5 mol propylene oxide to alkylphenols with 8 to 15 carbon atoms in alkyl groups thereof, C₁₂- to C₂₂-fatty acid monoesters of addition products of 1 to 30 mol ethylene oxide with glycerol; C₁₂- to C₂₂-fatty acid diesters of addition products of 1 to 30 mol ethylene oxide with glycerol, addition products of 5 to 60 mol of ethylene oxide with castor oil, addition products of 5 to 60 mol of ethylene oxide with hydrogenated castor oil, esters of phosphoric acid with addition products of 2 to 30 mol of ethylene oxide with C₈- to C₂₂-fatty alcohols, esters of sucrose with one or two fatty acids with 8 to 22 carbon atoms, esters of sorbitan with one, two or three fatty acids with 8 to 22 carbon atoms and with an ethoxylation degree of 4 to 20, polyglyceryl fatty acid esters of at least one fatty acid with 8 to 22 carbon atoms and polyglycerol with 2 to 20 glyceryl units, alkyl glycosides, C₈- to C₂₂-alkyltrimethylammonium compounds, C₈- to C₂₂-alkyldimethylhydroxyethylammonium compounds, di-(C₈- to C₂₂-alkyldimethyl ammonium compounds, C₈- to C₂₂-alkylpyridinium salts, C₈- to C₂₂-alkylamido ethyltrimethylammonium ether sulfates, C₈- to C₂₂-alkylmethylamine oxide, C₈- to C₂₂-alkylaminoethyldimethylamine oxide, amidoamines and quaternarized amidoamines.

19. The hair treatment composition as defined in claim 1, further comprising at least one hair dyeing pigment, and wherein said at least one hair dyeing pigment is selected from the group consisting of titanium dioxide (C.I. 77 891), black iron oxide (C.I. 77 499), yellow iron oxide (C.I. 77 492), red and brown iron oxide (C.I. 77 491), manganese violet (C.I. 77 742), ultramarine (C.I. 77 007), chromium oxide hydrate (C.I. 77 289), Iron Blue (C.I. 77 510), bismuth oxychloride (C.I. 77 163), carmine (cochineal) and pearlescent pigments based on mica that are coated with a metal oxide or oxychloride coating;
wherein said pearlescent pigments comprise titanium dioxide and/or bismuth oxychloride and other color-imparting materials, as needed, whose color is modified by changing thickness of the metal oxide or oxychloride coating; and
wherein said other color-imparting materials comprise iron oxides, iron blue, ultramarine and carmine.

20. The hair treatment composition as defined in claim 1, in the form of a hair gel in the form of a viscose hair lotion or in the form of a hair spray gel, which is sprayable by a mechanical spraying device, further comprising at least one thickening polymer and having a viscosity of at least 250 mPa s, as measured with a Bohlin Rheometer CS, measuring body C25 at 25° C. and a shear rate of 50 s¹.

21. The hair treatment composition as defined in claim 1, in the form of an oil-in-water emulsion, a water-in-oil emulsion or a microemulsion and further comprising at least one oil or wax and at least one emulsifier.

22. The hair treatment composition as defined in claim 1, in the form of a hair spray and wherein said hair spray includes at least one propellant or is formulated for a mechanical pump spraying apparatus; and wherein said at least one propellant is selected from the group consisting of propane, butane, dimethyl ether and fluorinated hydrocarbons.

23. The hair treatment composition as defined in claim 1, in the form of foamable product containing at least one foam-forming surfactant or at least one foam-forming polymer and wherein said foamable product includes at least one propellant or is formulated for a mechanical pump foaming apparatus; and wherein said at least one propellant is selected from the group consisting of propane, butane, dimethyl ether and fluorinated hydrocarbons.

24. The hair treatment composition as defined in claim 1, in the form of a solid hair wax and further comprising at least one wax selected from the group consisting of paraffin waxes, polyolefin waxes, wool wax, wool wax alcohol, candelilla wax, olive wax, carnauba wax, Japan wax, apple wax, hardened fats, fatty acid esters, fatty acid glycerides, fatty acid triglycerides, polyethylene glycol waxes and silicone waxes.

25. The hair treatment composition as defined in claim 1, in the form of a hair cleansing composition and further comprising from 0.01 to 40 percent by weight of at least one anionic, amphoteric or non-ionic hair-cleansing surfactant and containing from 50 to 90% water.

26. The hair treatment composition as defined in claim 1, in the form of a permanent shaping composition and further comprising at least one keratin-reducing substance and wherein said at least one keratin-reducing substance comprises a mercapto compound with keratin-reducing action.

27. The hair treatment composition as defined in claim 1, in the form of a hair bleaching composition, an oxidation hair dye composition or a permanent wave fixing agent, and further comprising at least one oxidizing agent.

28. The hair treatment composition as defined in claim 1, in the form of a hair coloring composition or an oxidation hair dye composition, and further comprising at least one synthetic or natural dyestuff absorbed directly on hair or at least one oxidation dye precursor compound.

29. A method of treating hair, said method comprising the steps of:
a) preparing a hair treatment composition according to one of claims 1 to 27;
b) applying the hair treatment composition to the hair; and
c) either allowing the hair treatment composition to remain on the hair or rinsing the hair treatment composition out of the hair after a predetermined acting time.
